(19) United States
(12) Patent Application Publication
(10) Pub. No.: US 2016/0138116 A1
(43) Pub. Date: May 19, 2016

(54) METHODS FOR MONITORING TREATMENT RESPONSE AND RELAPSE IN BREAST CANCER

(71) Applicant: AFG TECHNOLOGIES S.A.R.L., Luxembourg (LU)

(72) Inventor: Saroj Velamakanni, Cambridge (GB)

(21) Appl. No.: 14/900,914

(22) PCT Filed: Jun. 26, 2014
(86) PCT No.: PCT/EP2014/063626

§ 371 Date: Dec. 22, 2015

(30) Foreign Application Priority Data
Jun. 26, 2013 (GB) 1311389.9

(51) Int. Cl. C12Q 1/68; G01N 33/574

(52) U.S. Cl. CPC C12Q 1/6886; G01N 33/57415; C12Q 2600/156; C12Q 2600/154; C12Q 2600/106; C12Q 2600/112; G01N 2333/96486; G01N 2440/14

(57) ABSTRACT

The present invention provides a method for monitoring the response to treatment in a patient undergoing breast cancer therapy, comprising detecting the presence of a loss-of-function-related genetic alteration in the PAPPA gene or its regulatory or promoter sequences in a sample obtained from the patient wherein if the genetic alteration is present but its quantitative level decreases during treatment, this is indicative of response to therapy, whereas, if there is no change or upward change in its quantitative level during therapy, this is indicative of non-response to therapy. The invention also relates to the use of specific biological markers for monitoring for relapse, and aiding the screening, primary diagnosis and staging of breast cancer.

### FIELD OF THE INVENTION

This invention relates to the use of specific biological markers for aiding the screening, primary diagnosis and staging of breast cancer. The biological markers are also useful for monitoring response to treatment and detecting relapse.

### BACKGROUND OF THE INVENTION

Neoplasms and cancer are abnormal growths of cells. Cancer cells rapidly reproduce despite restriction of space, nutrients shared by other cells, or signals sent from the body to stop reproduction. Cancer cells are often shaped differently from healthy cells, do not function properly, and can spread into many areas of the body. Abnormal growths of tissue, called tumours, are clusters of cells that are capable of growing and dividing uncontrollably. Tumours can be benign (noncancerous) or malignant (cancerous). Benign tumours tend to grow slowly and do not spread. Malignant tumours can grow rapidly, invade and destroy nearby normal tissues, and spread throughout the body. Precursor lesions such as pre-invasive lesions or proliferative lesions with uncertain malignant potential represent abnormal growths of tissue which can behave in a benign fashion or alternatively progress to an invasive malignant cancer. Malignant cancers can be both locally invasive and metastatic.

Breast cancer is an example of a common cancer and is a complex disease due to its morphological and biological heterogeneity, its tendency to acquire chemo-resistance and the existence of several molecular mechanisms underline its pathogenesis. Half of women who receive loco-regional treatment for breast cancer will never relapse, whereas the other half will eventually die from metastatic disease. It is therefore imperative to distinguish clearly between those two groups of patients for optimal clinical management.

There is an urgent need for improved methods for monitoring patients' response to breast cancer treatment and monitoring for relapse, as well as new methods for aiding the screening, diagnosis and staging of breast cancer.

### SUMMARY OF THE INVENTION

The present invention is based on the finding that Pregnancy-Associated Plasma Protein A (PAPPA) is required for normal progression through mitosis, and that PAPPA silencing is highly prevalent in invasive breast cancer and pre-invasive lesions predisposed to becoming invasive. Therefore, the present invention provides a very important understanding to the biological causes of breast cancer, and allows consequent detection and treatment of breast cancer to be made in a more focussed and effective way. The understanding that PAPPA is required for normal progression through mitosis, and that the loss of its expression or impaired functioning contributes significantly to the cancerous state, allows detection of breast cancer to be made by monitoring PAPPA genetic alterations and/or expression or activity levels. The inventors have surprisingly found that PAPPA has utility as a blood-based marker of breast cancer.

According to a first aspect of the invention, there is a method for monitoring the response to treatment in a patient undergoing breast cancer therapy, comprising detecting the presence of a loss-of-function-related genetic alteration in the PAPPA gene or its regulatory or promoter sequences in a sample obtained from the patient wherein if the genetic alteration is present but its quantitative level decreases during treatment, this is indicative of response to therapy, whereas, if there is no change or upward change in its quantitative level during therapy, this is indicative of non-response to therapy.

According to a second aspect of the invention, there is a method for monitoring the response to treatment in a patient undergoing breast cancer therapy, comprising detecting the presence and/or level of PAPPA in a sample obtained from the patient, wherein the absence of PAPPA, or PAPPA activity, or the presence of PAPPA, or PAPPA activity at a reduced level compared to a control is indicative of non-response to therapy.

According to a third aspect of the invention, there is a method for determining whether a patient, previously treated with breast cancer therapy, has suffered a relapse, comprising detecting the presence of a loss-of-function-related genetic alteration in the PAPPA gene or its regulatory or promoter sequences in a biological sample obtained from the patient, wherein the presence and/or level of a genetic alteration is indicative of a relapse.

According to a fourth aspect of the invention, there is a method for determining whether a patient previously treated with breast cancer therapy, has suffered a relapse, comprising detecting the presence and/or level of PAPPA in a sample obtained from the patient, wherein the absence of PAPPA, or PAPPA activity, or the presence of PAPPA, or PAPPA activity at a reduced level compared to a control, is indicative of a relapse.

According to a fifth aspect of the invention, there is a method for determining whether a patient previously treated with breast cancer therapy has suffered a relapse, comprising identifying the proportion of mitotic cells in a tissue sample obtained from the patient that are in prophase or pro-metaphase and comparing to a pre-determined cut-off value, wherein if the proportion of cells in prophase or pro-metaphase is the same or greater than the cut-off value there is a risk that the patient has suffered a relapse.

According to a sixth aspect, there is a method for determining whether a primary breast cancer in a patient has spread away from the primary tumour to other parts of the body or infiltrated tissue adjacent to the primary tumour, comprising detecting the presence of a loss-of-function-related genetic alteration in the PAPPA gene or its regulatory or promoter sequences in a sample obtained from the patient, wherein the presence of a genetic alteration is indicative of spread or infiltration, and wherein the sample is a blood sample or tissue adjacent to or distant from the primary tumour site.

According to a seventh aspect, there is a method for determining whether a primary breast cancer in a patient has spread away from the primary tumour to other parts of the body or infiltrated tissue adjacent to the primary tumour, comprising identifying the proportion of mitotic cells in a tissue sample obtained from the patient that are in prophase or pro-metaphase and comparing to a pre-determined cut-off value, wherein if the proportion of cells in a prophase or pro-metaphase is the same or greater than the cut-off value there is a risk that the cancer has spread or infiltrated, wherein the sample is from tissue adjacent to or distant from the primary tumour site.

According to an eighth aspect, there is a method for determining whether a primary breast cancer in a patient has spread away from the primary tumour to other parts of the body or infiltrated tissue adjacent to the primary tumour, comprising detecting the presence and/or level of PAPPA in a sample obtained from the patient, wherein the absence of PAPPA, or PAPPA activity or the presence of PAPPA, or PAPPA activity, at a reduced level compared to a control, is indicative of spread or infiltration, wherein the sample is a blood sample or a tissue sample adjacent to or distant from the site of the primary tumour.

According to a ninth aspect, there is a method for screening for breast cancer in a subject, comprising detecting the presence of a loss-of-function-related genetic alteration in the PAPPA gene or its regulatory or promoter sequences in a blood sample obtained from the subject, wherein the presence of a genetic alteration is indicative of breast cancer.

According to a tenth aspect, there is a method for screening for breast cancer in a subject, comprising detecting the presence and/or level of PAPPA in a blood sample obtained from the subject, wherein the absence of PAPPA or PAPPA activity in the sample, or the presence of PAPPA, or PAPPA activity, at a reduced level compared to a control, is indicative of breast cancer.

According to an eleventh aspect, there is a method for aiding primary diagnosis of breast cancer in a patient, comprising detecting the presence of a loss-of-function-related genetic alteration in the PAPPA gene or its regulatory or promoter sequences in a sample obtained from the patient, wherein the presence of a genetic alteration is indicative of breast cancer, and wherein the sample is selected from tissue, blood and nipple aspirates.

According to a twelfth aspect, there is a method for aiding primary diagnosis of breast cancer in a patient, comprising detecting the presence and/or level of PAPPA, or PAPPA activity, in a sample obtained from the patient, wherein if PAPPA, or PAPPA activity, is not present, or is present at a reduced level compared to a control, the result is indicative of breast cancer, and wherein the sample is selected from tissue, blood and nipple aspirates.

### DESCRIPTION OF THE DRAWINGS

The invention is described with reference to the accompanying figures, wherein:

FIG. 1 shows mitotic phase distribution in human cancers. 1a shows representative images identifying distinct mitotic phases by H3S10ph immunolabelling in tissue sections of surgical biopsy specimens (1000× magnification). 1b shows pie charts showing the percentage of mitotic cells assigned to each mitotic phase in normal breast (n=5 patients), lymphoma (n=29 patients) and in breast cancer (n=156 patients), lung cancer (n=30 patients), bladder cancer (n=27 patients) and colon cancer (n=41 patients). 1c shows representative cases of breast cancer (400× magnification; scale bar 50 μm) and non-invasive ductal carcinoma in situ (DCIS; 400× magnification; scale bar 50 μm) which show a high frequency of early mitotic figures compared to bladder cancer (1000× magnification; scale bar 20 μm) and other cancer types (not shown);

FIG. 2 shows specificity of phosphohistone H3 (H3S10ph) as a mitotic marker. HeLa Kyoto cells were synchronised at the G1/S transition by double-thymidine block and in prometaphase by treatment with the Plk-1 inhibitor BI2536 (SelleckChem) at 5 μM. Asynchronously proliferating (UT), thymidine-arrested, and BI2536-treated cells were immuno-labelled for phosphohistone H3 (H3S10ph). Phosphohistone H3 was not detected in thymidine-arrested cells by immunofluorescence or chromogenic staining, whereas BI2536-treated showed an enrichment of prometaphase cells (arrows) positive for H3S10ph. Panels on the right show flow cytometric analysis of DNA content;

FIG. 3 is a Receiver Operating Characteristic curve for prophase/prometaphase fraction applying a minimum mitotic cell count of n=5;

FIG. 4 shows the distribution of prophase/prometaphase fraction in breast cancer, DCIS and other cancers (pooled);

FIG. 5 shows enrichment of early mitotic figures in breast cancer. 5a is a box plot showing the percentage of mitotic cells in prophase/prometaphase in a range of human cancers. Breast cancer is characterised by a higher proportion of mitotic cells in prophase/prometaphase compared to other tumour types (P<0.0001). The median (solid black line), interquartile range (boxed) and range (enclosed by lines) are shown. Outlying cases are depicted as isolated points. 5b shows photomicrographs of representative cases of normal breast, breast cancer and other types of cancer immuno-labelled for phosphohistone H3 (H3S10ph) and assigned to distinct mitotic phases (see key below; 1000× magnification, scale bar 20 μm);

FIG. 6 shows that acquisition of the mitotic delay phenotype occurs early in multi-step mammary tumour progression. 6a shows photomicrographs of representative cases of non-invasive ductal carcinoma in situ (DCIS) immuno-labelled for phosphohistone H3 (H3S10ph) showing normal mitotic phase distribution (left panel) and a high proportion of mitotic cells in prophase/prometaphase (right panel) (1000× magnification; scale bar 20 μm). 6b is a bar chart showing the percentage of cases of normal breast, DCIS and breast cancer exhibiting prophase/prometaphase delay. Cases are defined as delayed if the proportion of mitotic cells in prophase/prometaphase is at least one third. This cut-point was chosen to allow the proportion of specimens in the combined group of other malignancies properly classified as non-delayed (94.1%) to be approximately equal to the proportion of breast cancer specimens properly classified as delayed (94.9%). 6c is a box plot showing the percentage of mitotic cells in prophase/prometaphase in normal breast, DCIS and breast cancer. There is a trend for increasing early mitotic delay during transition from normal breast to invasive breast cancer (P<0.001);

FIG. 7 shows selection of MitoCheck prophase/prometaphase class genes for further study. The genome-wide MitoCheck RNAi screen was performed by time-lapse fluorescence microscopy of live HeLa Kyoto cells stably expressing a fluorescent chromosome marker (Histone 2B-GFP) (1). Automated fluorescence imaging of siRNA transfected cells was followed by computational phenotyping of mitotic stages and mitotic defects from digital images (2). The time-resolved phenoprints were analysed to cluster candidate genes by phenotype (3). Data mining of the MitoCheck prophase/prometaphase class genes revealed 41 genes linked to early mitotic phase progression (4, 5). Several exclusion criteria (for example, no massive cell death as a secondary phenotype) were used to narrow the list of candidates to seven genes whose knock down caused a sharp increase in the percentage of mitotic cells in prophase/prometaphase (6, 7);

FIG. 8a shows time-resolved heat maps for seven candidate genes identified in the genome-wide MitoCheck screen in HeLa cells, which show as a primary phenotype prometaphase arrest/delay followed by secondary phenotypes (binuclear, polylobed, grape-shaped and cell death). FIG. 8b shows that knock down of all seven candidate genes caused a significant increase in the percentage of mitotic cells in prophase/prometaphase;

FIG. 9 shows PAPPA silencing through promoter methylation is linked to mitotic delay in breast cancer. 9a is a heat map showing the promoter methylation status (determined by MethyLight assay as percentage methylated reference gene [PMR]) of the seven MitoCheck candidate genes in normal breast (n=30 patients), low-grade (n=39 patients) and high-grade (n=36 patients) non-invasive DCIS breast lesions, and in invasive breast cancer (n=173 patients). PMR values are presented by coloured bars as shown in the key below the panel. 9b is a stacked bar chart showing normal breast (n=30 patients), DCIS (n=75 patients) and breast cancer (n=173 patients) cases ranked by PAPPA promoter methylation level. 9c shows PAPP-A protein expression in normal proliferating (pregnant) breast, DCIS and breast cancer cases in relation to mitotic delay phenotype and PAPPA promoter methylation status (1000× magnification; scale bar 10 μm). PAPPA antibody specificity was confirmed by peptide blocking (lower right panel). 9d is a heat map showing the promoter methylation status of the seven MitoCheck candidate genes in cultured primary, immortalised, and transformed breast cells. 9e shows detection of PAPPA protein by western blot in the cultured breast cells described in panel (d). 9f is a stacked bar chart showing the percentage of mitotic cells in the cultured breast cells described in panel (d) assigned to distinct mitotic phases;

FIG. 10 shows characterisation of rabbit polyclonal antibody raised against PAPPA. 10a is a Western blot detection of endogenous PAPPA in whole cell extracts prepared from MCF10A and BT549 cells. Pre-incubation with a blocking peptide confirmed the specificity of the PAPPA antibody, while the pre-immune serum did not detect endogenous PAPPA. 10b is a Western blot analysis of whole cell extracts prepared from untreated (UT), ZMPSTE24 overexpressing (CO) and PAPPA overexpressing (PAPPA+) T47D cells with PAPPA antibody. Notably T47D cells show PAPPA promoter hypermethylation and do not express the endogenous protein. 10c shows immunoprecipitation of PAPP-A protein from cell culture medium obtained from PAPPA overexpressing T47D cell populations (PAPPA+) 72 hour post-transfection. PAPPA protein was immuno-precipitated with a commercially supplied PAPPA rabbit polyclonal antibody (DAKO) and detected by Western blot using the in-house raised PAPPA antibody. FT: flow-through following overnight incubation with antibody coated beads; Elution: bound proteins eluted from beads with loading buffer;

FIG. 11 shows experimental manipulation of PAPPA expression controls transit through early mitosis. 11a shows PAPPA transcript levels were knocked down (KD) by RNAi in BT549 cells, resulting in PAPPA protein depletion compared to untreated (UT) and control-transfected (CO) cells. 11b shows PAPP-A protein levels were restored in T47D cells (PAPPA gene epigenetically silenced by promoter methylation) after transfection with a PAPPA expression construct (PAPPA+). T47D cells were control-transfected (CO) with an expression construct for an unrelated metalloproteinase (ZMPSTE24), which—like PAPPA—is a member of the metzincin family. 11c shows RNAi against PAPPA in BT549 cells was associated with a marked increase in early mitotic figures as determined by phosphohistone H3 (H3S10ph) immuno-labelling of cytospin preparations, while exogenously expressed PAPPA restored normal mitotic phase distribution in T47D cells. 11d shows the indicated time points cell number was measured in UT, CO and KD BT549 cells. 11e shows the DNA content of UT, CO and KD BT549 cells 48 and 72 hours post-transfection.

FIG. 12 shows RNAi specificity control and rescue experiments in BT549 breast cancer cells. 12a shows PAPPA transcript levels in untreated (UT) cells, PAPPA-siRNA 104028 (KD28) or PAPPA-siRNA 10042 (KD42) transfected cells, and in cells transfected with PAPPA RNAi rescue construct in the presence of PAPPA siRNA (PAPPA+^{mut}/KD28 or PAPPA+^{mut}/KD42) relative to cells transfected with a ZMPSTE24 expression construct (CO). 12b is a Western blot analysis of PAPP-A protein in whole cell extracts prepared from UT, CO, KD28, KD42, PAPPA+^{mut}/KD28 and PAPPA+^{mut}/KD42 cells 72 hours post-transfection. 12c shows UT, CO, KD28, KD42, PAPPA+^{mut}/KD28 and PAPPA+^{mut}/KD42 cells were cytospun onto glass slides and mitotic cells were detected by phosphohistone H3 (H3S10ph) immuno-labelling. 12d is a stacked bar chart showing the percentage of UT, CO, KD28, KD42, PAPPA+^{mut}/KD28 and PAPPA+^{mut}/KD42 cells in distinct mitotic phases;

FIG. 13 shows PAPPA expression levels affect the invasive capacity of breast cancer cell lines. 13a shows that PAPPA expression in BT549 and T47D cells was experimentally manipulated as described in the legend to FIG. 11 and the invasiveness of the cells measured in Boyden Chamber assays. 13b shows crystal violet stained Boyden Chamber inserts for PAPPA depleted (KD) BT549 cells and PAPPA overexpressing (PAPPA+) T47D cells compared to untreated (UT) and control-transfected (CO) cells. 13c shows surface β1-integrin levels in CO and KD BT549 cells. 13d shows the increased invasiveness associated with PAPPA depletion in BT549 cells was reversed by addition of an anti-β1-integrin blocking antibody to the culture medium for the duration of the invasion assay;

FIG. 14 shows that the PAPPA promoter is methylated in circulating tumour DNA isolated from breast cancer patients, whereas no methylation is detectable in healthy controls. (A) shows a representative amplification plot of a MethyLight assay for methylated PAPPA promoter using DNA purified from plasma of breast cancer patients (n=3) compared to a healthy control. The relative fluorescence (Rn) of one representative sample well is plotted against the number of PCR amplification cycles. (B) shows a MethyLight assay amplification of Col2A1 DNA that was used as a reference to estimate the DNA amount isolated from each sample;

FIG. 15 shows the percentage of breast cancer cases (n=12) exhibiting PAPPA promoter hypermethylation in circulating tumour DNA compared to normal controls. Positive PAPPA methylation was defined as a Ct<45 cycles (p=0.022 for breast cancer versus healthy control cases, Fisher's exact test); and

FIG. 16 shows the distribution of the detectable amount of PAPPA promoter methylation between breast cancer samples. The distribution of the Ct values (obtained from the MethyLight assay) against the methylated PAPPA promoter are shown. The figure shows a comparison between circulating DNA isolated from control plasma samples (n=3, solid bars) and breast cancer samples (n=12, unfilled bars). The x-axis denotes the minimum of the Ct value range, while the y-axis denotes the frequency of samples in the range. In cases with no detectable amplification, the Ct is displayed as N/A.

### DESCRIPTION OF THE INVENTION

The following definitions apply to terms used throughout this description and in relation to any of the aspects of the invention described herein.

The term “patient” refers to any animal (e.g. mammal), including, but not limited to, humans, non-human primates, canines, felines, rodents and the like, which is to be the recipient of the diagnosis. Typically, the term “patient” is used herein in reference to a human subject.

The terms “cancer” and “cancerous” refer to or describe the physiological condition in mammals in which a population of cells are characterised by unregulated cell growth.

The terms “cancer cell” and “tumour cell” are grammatical equivalents referring to the total population of cells derived from a tumour or a pre-cancerous lesion.

The term “breast cancer” includes all forms of primary breast carcinoma, including invasive ductal carcinoma, invasive lobular carcinoma, tubular carcinoma, medullary carcinoma, alveolar carcinoma, solid variant carcinoma, signet ring cell carcinoma, metaplastic carcinoma.

The term “invasive cancer” refers to cancer that has spread beyond the primary tumour site in which it developed, and is growing in surrounding, healthy tissues. Invasive cancer is sometimes referred to as infiltrating cancer. The term is intended to include all primary invasive breast cancers including, invasive ductal carcinoma “not otherwise specified” (IDC) and IDC subtypes (e.g. mixed, pleomorphic, osteoclast types), invasive lobular carcinoma (ILC), tubular carcinoma, mucinous carcinoma, medullary carcinoma, neuroendocrine tumours, invasive papillary and cribriform carcinoma and invasive apocrine, metaplastic and oncocytic subtypes.

As used herein, the term “remission” has its usual meaning in the art, and refers to a state of absence of detectable disease in a subject who has previously been treated for cancer.

The terms “drug” and “agent” are used interchangeably herein, and refer to a chemical or biological substance that exerts an effect on a biological system.

The present invention can utilise naturally-occurring or synthetic nucleic acids. The methods described herein can involve the synthesis of cDNA from mRNA in a test sample or the amplification of naturally-occurring nucleic acids. Portions of cDNA corresponding to genes or gene fragments of interest can be amplified, and the product detected.

As used herein, the term “a sample” includes biological samples obtained from a patient or subject, which may comprise a tissue sample, or liquid/fluid biopsy samples, including blood/blood components and nipple aspirates.

Liquid biopsy is advantageous over tissue biopsy, because it is less invasive to obtain a liquid sample from the patient or subject, and liquid biopsy overcomes some of the issues of tumour heterogeneity associated with tissue biopsy; information acquired from a single biopsy provides a spatially and temporally limited snap-shot of a tumour that does not necessarily reflect its heterogeneity. A liquid biopsy can provide the genetic landscape of all cancerous lesions (primary and metastases) as well as offering the opportunity to systemically track genomic evolution (Crowley, E. et al., Nat. Rev. Clin. Oncol. 2013).

As used herein, the term “blood sample” includes blood components, including plasma and serum. Circulating free DNA, mRNA, miRNA and, if present, circulating tumour cells, can be extracted from the blood sample and used to determine the PAPPA status of the subject. The skilled person will be familiar with standard phlebotomy techniques which are suitable for obtaining a blood sample from a subject.

As used herein, the term “nipple aspirates” refers to breast fluid obtained from the nipple of a non-lactating woman. It is also referred to as nipple aspirate fluid, (NAF). The fluid is collected from the nipple by gentle aspiration and contains cells and extracellular fluid from the breast ductal epithelium.

Examples of suitable tissue samples include formalin-fixed paraffin-embedded (FFPE) biopsy and/or resection specimens. These comprise protein, DNA, mRNA and/or miRNA, other cellular and extracellular matter and tumour cells (if present in the subject). In the context of the present invention, tissue samples can be used to determine the PAPPA status of the subject (i.e. determine PAPPA expression/activity levels and presence of loss-of-function genetic alterations) and to identify the mitotic delay phenotype by analysing mitotic phase distribution in dividing cells within the tissue. Methods for taking a sample from a patient (biopsy) are conventional and will be apparent to the skilled person.

Preferably the tissue sample obtained from the patient and used in the in vitro methods of the invention may be a breast tissue sample. The breast tissue sample may exhibit proliferative lesions or it may be cancerous tissue. In some aspects of the invention relating to staging of breast cancer, the tissue sample may be taken from a site that is adjacent to or distant from the site of a primary breast tumour.

As used herein, the term “proliferative lesions” refers to lesions with atypia. Proliferative lesions with atypia represent precursor lesions of invasive breast cancer. Precursor lesions can be divided broadly into two groups, namely “pre-invasive lesions” and “proliferative lesions with uncertain malignant potential”. These two groups of entities represent proliferation of atypical or malignant cells within the breast parenchymal structures but in which there is no evidence of invasion across the basement membrane. Pre-invasive lesions include ductal carcinoma-in-situ (DCIS), lobular carcinoma-in-situ (LCIS) and Paget's disease of the nipple. Proliferative lesions with uncertain malignant potential include such entities as lobular neoplasia, lobular intraepithelial neoplasia, atypical lobular hyperplasia (ALH), flat epithelial atypia (FEA), atypical ductal hyperplasia (ADH) microinvasive carcinoma, intraductal papillary neoplasms and phyllodes tumour. These entities are well characterised in the art and used in routine clinical pathological practice.

The methods of the invention described herein are carried out in vitro. For the avoidance of doubt, the term “in vitro” has its usual meaning in the art, referring to methods that are carried out in or on tissue in an artificial environment outside the body of the patient from whom the tissue sample has been obtained.

The terms “immunoassay”, “immuno-detection” and “immunological assay” are used interchangeably herein and refer to antibody-based techniques for identifying the presence of or levels of a protein in a sample.

The term “antibody” refers to an immunoglobulin which specifically recognises an epitope on a target as determined by the binding characteristics of the immunoglobulin variable domains of the heavy and light chains (V_{H}S and V_{A}S), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example F_{ab}, F_{ab}′, and Fᵣ fragments, linear antibodies, single chain antibodies, and multispecific antibodies comprising antibody fragments), single chain variable fragments (scFᵥS), multispecific antibodies, chimeric antibodies, humanised antibodies and fusion proteins comprising the domains necessary for the recognition of a given epitope on a target. Antibodies may also be conjugated to various moieties for a diagnostic effect, including but not limited to radionuclides, fluorophores or dyes.

The term “specifically recognises”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition.

The term “mitosis” has its usual meaning in the art. Mitosis is the process by which a eukaryotic cell separates the chromosomes in its cell nucleus into two identical sets, in two separate nuclei. Mitosis and cytokinesis together define the mitotic (M) phase of the cell cycle. The process of mitosis is characterised into stages corresponding to the completion of one set of activities and the start of the next. These stages are prophase, prometaphase, metaphase, anaphase and telophase.

The term “prophase” has its usual meaning in the art. Prophase refers to the stage where the chromatin in the nucleus becomes tightly coiled, condensing into discrete chromosomes.

The term “prometaphase” has its usual meaning in the art. During prometaphase the nuclear membrane disintegrates and microtubules invade the nuclear space. The present inventors have identified that suppression of endogenous PAPPA levels is implicated in the development of breast cancer.

This invention enables methods to be developed for aiding the screening, primary diagnosis and staging of breast cancer, in addition to aiding the detection of relapse and monitoring of treatment response, by detecting the presence/absence of the Pregnancy-Associated Plasma Protein A (PAPPA) or the presence of loss-of-function alterations in the PAPPA gene or its promoter sequence.

PAPPA was identified in 1974 as one of four proteins of placental origin circulating at high concentrations in pregnant women, and later found clinical utility as a biomarker for Down's syndrome pregnancies. Its biological function remained an enigma for a quarter of a century until it was identified as a protease that regulates IGF bioavailability through cleavage of the inhibitory insulin-like growth factor binding protein-4 (IGFBP-4). Its role as an IGFBP-4 protease in a diverse range of cell types (e.g. fibroblasts, osteoblasts and vascular smooth muscle cells), together with a highly conserved amino acid sequence in vertebrates, indicated that PAPPA serves a basic function beyond placental physiology.

Mitotic delay due to PAPPA suppression in breast cancer cells at first glance appears disadvantageous to tumour growth. However a major biological advantage is conferred to the mitotically delayed, neoplastic breast cell through the associated increase in acquiring invasive capacity. In breast cancer specimens, mitotic delay linked to PAPPA silencing can be detected in virtually all cases of invasive cancer and also in a proportion of non-invasive lesions. The gain in invasive capacity as a consequence of PAPPA loss therefore occurs early in multi-step mammary tumour progression during the transition from non-invasive to invasive cancer. Detection of PAPPA deregulation and mitotic delay in clinical biopsy specimens offers a significant advance in the detection of breast cancer and management of breast cancer patients.

The present invention is useful in the context of:

1) Monitoring a patient's response to a breast cancer treatment

2) Detecting a relapse after treatment

3) Staging of breast cancer

4) Screening patients to detect early invasive or pre-invasive breast cancer

5) Primary diagnosis of breast cancer

6) Prediction of disease progression of invasive breast cancer

### 1. Monitoring Response to Treatment

The present invention is useful for monitoring a patient's response to a treatment given for breast cancer.

Conventional treatments for breast cancer include surgery, sentinel lymph node biopsy followed by surgery, radiation therapy, chemotherapy, hormone therapy and targeted therapy.

The present invention provides a way of monitoring the success of the therapy and monitoring progress during therapy.

According to a first aspect of the invention, there is a method for monitoring the response to treatment in a patient undergoing breast cancer therapy, comprising detecting the presence of a loss-of-function-related genetic alteration in the PAPPA gene or its regulatory or promoter sequences in a sample obtained from the patient, wherein if the genetic alteration is present but its quantitative level decreases during treatment, this is indicative of response to therapy, whereas if there is no change or upward change in its quantitative level this is indicative of non-response to therapy.

Detecting genetic alterations is useful in monitoring the response to therapy. If there is no change in the level of the mutant gene this indicates non-response. If there is a gradual decrease in the level of the mutant gene, then the patient's tumour is responding to treatment. If the mutant gene cannot be detected any longer it indicates that the patient is in remission.

The quantitative level of PAPPA genetic alterations in a patient's sample is tested at intervals during the therapeutic treatment, using a technique that generates a quantitative readout (e.g. digital PCR and cold PCR). If the tumour cells are responding to the treatment, results with decreasing signal strength will be generated (i.e. a downward change in the quantitative level of PAPPA genetic alterations). If the tumour cells are resistant to the chosen therapy and continue to divide, then the signal strength will increase (i.e. an upward change in the quantitative level of PAPPA genetic alterations). If the quantitative level of PAPPA genetic alterations stays the same during the therapeutic treatment then this is indicative of stable disease.

In a preferred embodiment, the loss-of-function-related genetic alteration in the PAPPA gene or its regulatory or promoter sequences is one or more of methylation, deletion, a point mutation, loss-of-heterozygosity, translocation, insertion or chromosomal breakage. Hypermethylation and deletion are believed to be the most relevant genetic alterations in this context.

According to a second aspect, there is a method for monitoring the response to treatment in a patient undergoing breast cancer therapy, comprising detecting the presence and/or level of PAPPA in a biological sample obtained from the patient, wherein the absence of PAPPA, or the presence of PAPPA at a reduced level compared to a control is indicative of non-response to therapy.

Preferably the control is the patient's baseline PAPPA levels determined from a sample taken from the patient prior to treatment commencing.

Monitoring for the presence of PAPPA, or PAPPA activity, will aid in the monitoring of the effectiveness of therapy, as the PAPPA levels may initially increase at the start of therapy, but, if the patient is non-responsive to therapy, the PAPPA levels may decrease. A rise in levels of PAPPA compared with the patient's baseline control sample and subsequent maintenance of PAPPA levels at those of or similar to a healthy control will indicate a successful response to treatment.

In a preferred embodiment, both of the above methods are carried out.

As used herein, the phrase “patient undergoing breast cancer therapy” includes both patients who have begun, but not yet completed, a course of therapy, in which case the method is carried out during therapy, and patients who have completed the course of therapy, in which case the method is carried out following therapy. In the latter instance, the methods of the invention are useful for indicating or confirming that a patient is in remission. The patient may be being treated for primary or metastatic breast cancer.

Typically the methods will be carried out using liquid biopsy samples, including blood samples and/or nipple aspirates. Preferably, the sample is one or more blood samples taken from the patient undergoing therapy.

The therapy may include any conventional breast cancer therapy, including any of those described herein.

### 2. Relapse Monitoring

The present invention may also be used to monitor a patient who is in complete or partial remission following treatment for primary or metastatic breast cancer, to determine whether there is a relapse or progressive disease. The present invention can therefore be used to provide early warning of relapse. The present invention is also useful in this context to help in the treatment of relapse.

As used herein, the term “relapse” has its usual meaning in the art, and refers to cancer returning, either in the breast or at another site (i.e. metastatic cancer) following complete remission.

The term “complete remission” (also known as “pathologic remission”) refers to the situation where no tumour cells are detectable following surgical removal of the cancerous tissue and chemotherapy.

“Partial remission” refers to a situation where the breast tumour has shrunk in size following chemotherapy but subsequent surgery to remove the remaining tumour is not possible (for example, due to the location of the tumour). This results in a stable disease state, where the tumour is present but not growing.

The term “progressive disease” refers to renewed tumour growth following a period of stable disease in a patient in partial remission.

According to this third aspect of the invention, there is a method for determining whether a patient, previously treated with breast cancer therapy, has suffered a relapse or progressive disease, comprising detecting the presence of a loss-of-function-related genetic alteration in the PAPPA gene or its regulatory or promoter sequences in a biological sample obtained from the patient, wherein the presence of a genetic alteration is indicative of a relapse.

In a preferred embodiment, the loss-of-function-related genetic alteration in the PAPPA gene or its regulatory or promoter sequences is one or more of methylation, deletion, a point mutation, loss-of-heterozygosity, translocation, insertion or chromosomal breakage. Hypermethylation and deletion are believed to be the most relevant genetic alterations in this context.

According to a further fourth aspect, there is a method for determining whether a patient previously treated with breast cancer therapy, has suffered a relapse or progressive disease, comprising detecting the presence and/or level of PAPPA in a sample obtained from the patient, wherein the absence of PAPPA, or the presence of PAPPA at a reduced level compared to a control, is indicative of a relapse.

Preferably the control is the patient's baseline PAPPA levels determined from a sample taken from the patient following completion of treatment.

According to a further fifth aspect, there is a method for determining whether a patient previously treated with breast cancer therapy has suffered a relapse or progressive disease, comprising identifying the proportion of mitotic cells in a biological sample obtained from the patient that are in prophase or pro-metaphase and comparing to a pre-determined cut-off value, wherein if the proportion of cells in prophase or pro-metaphase is the same or greater than the cut-off value there is a risk that the patient has suffered a relapse.

In the methods of the third and fourth aspects of the invention, the sample from the patient is typically blood, nipple aspirates or tissue sample. Preferably, the sample is a blood sample.

In the analysis of mitotic delay according to the method of the fifth aspect of the invention, the sample from the patient may be tissue or nipple aspirates

In these aspects of the invention, the patient will have been previously treated for breast cancer. For the avoidance of doubt, the phrase “previously treated” means that the patient will have been diagnosed with breast cancer and will have completed the prescribed treatment for the breast cancer and entered complete or partial remission before the methods of any of the third to fifth aspects of the invention are carried out. The patient may have been treated for primary or metastatic breast cancer.

Any or all of the three aspects mentioned above may be combined in order to monitor for relapse.

### 3. Staging

After breast cancer has been diagnosed, tests are done to find out if cancer cells have infiltrated within the breast or spread to other parts of the body.

The process used to find out whether the cancer has infiltrated within the breast or spread to other parts of the body is called staging. The information gathered from the staging process determines the stage of the disease and enables differential diagnosis of different tumour types (e.g. primary versus metastatic or secondary tumours). It is important to know the stage in order to plan treatment. The following tests and procedures may be used in the staging process:
Blood test and breast MRI
Sentinel lymph node biopsy: The removal of the sentinel lymph node during surgery. The sentinel lymph node is the first lymph node to receive lymphatic drainage from a tumour. It is the first lymph node the cancer is likely to spread to from the tumour. A radioactive substance and/or blue dye is injected near the tumour. The substance or dye flows through the lymph ducts to the lymph nodes. The first lymph node to receive the substance or dye is removed. A pathologist views the tissue under a microscope to look for cancer cells. If cancer cells are not found, it may not be necessary to remove more lymph nodes.
Chest x-ray: An x-ray of the organs and bones inside the chest. An x-ray is a type of energy beam that can go through the body and onto film, making a picture of areas inside the body.
CT scan (CAT scan): A procedure that makes a series of detailed pictures of areas inside the body, taken from different angles. The pictures are made by a computer linked to an x-ray machine. A dye may be injected into a vein or swallowed to help the organs or tissues show up more clearly. This procedure is also called computed tomography, computerized tomography, or computerized axial tomography.
Bone scan: A procedure to check if there are rapidly dividing cells, such as cancer cells, in the bone. A very small amount of radioactive material is injected into a vein and travels through the bloodstream. The radioactive material collects in the bones and is detected by a scanner.
PET scan (positron emission tomography scan): A procedure to find malignant tumour cells in the body. A small amount of radioactive glucose (sugar) is injected into a vein. The PET scanner rotates around the body and makes a picture of where glucose is being used in the body. Malignant tumour cells show up brighter in the picture because they are more active and take up more glucose than normal cells do.

The three ways that cancer spreads in the body are:

1. Through tissue—cancer invades the surrounding normal tissue.
2. Through the lymph system—cancer invades the lymph system and travels through the lymph vessels to other places in the body.
3. Through the blood—cancer invades the veins and capillaries and travels through the blood to other places in the body.

When cancer cells break away from the primary (original) tumour and travels through the lymph or blood to other places in the body, another (secondary) tumour may form. This process is called metastasis. The secondary (metastatic) tumour is the same type of cancer as the primary tumour. For example, if breast cancer spreads to the bones, the cancer cells in the bones are actually breast cancer cells. The disease is metastatic breast cancer, not bone cancer.

As used herein, the term “infiltrate” refers to local invasion of tumour cells to tissue within the breast that is adjacent to the primary tumour site.

The present invention can be used separately, or as an additional test complementing conventional staging techniques, to stage the cancer, and determine whether a primary breast cancer has infiltrated adjacent breast tissue or spread to distant tissue.

According to this sixth aspect of the invention, there is a method for determining whether a primary breast cancer in a patient has spread away from the primary tumour to other parts of the body or infiltrated tissue adjacent to the primary tumour, comprising detecting the presence of a loss-of-function-related genetic alteration in the PAPPA gene or its regulatory or promoter sequences in a sample obtained from the patient, wherein the presence of a genetic alteration is indicative of infiltration or spread.

In a preferred embodiment, the loss-of-function-related genetic alteration in the PAPPA gene or its regulatory or promoter sequences is one or more of methylation, deletion, a point mutation, loss-of-heterozygosity, translocation, insertion or chromosomal breakage. Hypermethylation and deletion are believed to be the most relevant genetic alterations in this context.

According to a further seventh aspect, there is a method for determining whether a primary breast cancer in a patient has spread away from the primary tumour to other parts of the body or infiltrated tissue adjacent to the primary tumour, comprising identifying the proportion of mitotic cells in a biological sample obtained from the patient that are in prophase or pro-metaphase and comparing to a pre-determined cut-off value, wherein if the proportion of cells in a prophase or pro-metaphase is the same or greater than the cut-off value there is a risk that the tumour has infiltrated or spread.

According to a further eighth aspect, there is a method for determining whether a primary breast cancer in a patient has spread away from the primary tumour to other parts of the body or infiltrated tissue adjacent to the primary tumour, comprising detecting the presence and/or level of PAPPA in a sample obtained from the patient, wherein the absence of PAPPA, or the presence of PAPPA at a reduced level compared to a control, is indicative of infiltration or spread. Preferably “control” refers to PAPPA levels measured in a sample from a healthy individual.

In each of the sixth and eighth aspects of the invention, the patient sample can be a blood sample, ascites, pleural effusion, or a tissue sample. Preferably the sample is a blood sample.

In the seventh aspect of the invention, the patient sample will typically be a tissue sample.

Tissue biopsy samples may be taken from breast tissue at a site adjacent to the site of the primary tumour or may be taken from a different site that is distant from (i.e. not at or adjacent to) the primary tumour site, e.g. skin, lymph nodes outside of the breast, lung or liver tissue.

The patient will have been diagnosed with breast cancer prior to these methods being carried out. Other tests may be performed on the patient to aid in the staging of the cancer. For example, one or more of sentinel lymph node biopsy, chest x-ray, CT (CAT) scan, bone scan, PET scan, may have been performed on the patient.

The results of the staging diagnosis can help in the prognosis (prediction of outcome) and in determining what further therapeutic intervention should be given.

The methods and techniques for carrying out the above aspects are as disclosed herein.

### 4. Screening

The present invention can also be used in the routine screening for breast cancer. As used herein, the term “screening” refers to the process of routinely testing an individual to detect the presence of a disease, in particular breast cancer. The subject is usually an individual who has presented themselves for routine screening for breast cancer, but who has not experienced or reported any symptoms of breast cancer or been motivated to seek medical attention due to symptoms of breast cancer (i.e. the subject is asymptomatic for breast cancer). Alternatively or additionally, the individual may be at high risk of breast cancer, for example due to family history of breast cancer.

The invention can be used separately, or preferably, as an additional test complementing conventional screening procedures. Screening tests can help find cancer at an early stage, before symptoms appear. When abnormal tissue or cancer is found early, it may be easier to treat or cure. By the time symptoms appear, the cancer may have grown and spread. This can make the cancer harder to treat or cure.

Conventional screening tests include the following:
Physical exam and history: Examination of the body to check general signs of health, including checking for signs of disease, such as lumps or anything else that seems unusual. A history of the patient's health habits and past illnesses and treatments will also be taken.
Laboratory tests: Medical procedures that test samples of tissue, blood, urine, or other substances in the body.
Imaging procedures: Procedures that make pictures of areas inside the body.
Genetic tests: Tests that look for certain gene mutations (changes) that are linked to some types of cancer.

One difficulty is that screening test results may appear to be abnormal even though there is no cancer. A false-positive test result (one that shows there is cancer when there actually is not) can cause anxiety and is usually followed by more tests and procedures, which also have risks.

The present invention can therefore be used to screen a patient and determine whether the patient has early invasive breast cancer and therefore requires further tests for diagnosis.

According to this ninth aspect of the invention, there is a method for screening for breast cancer in a subject, comprising detecting the presence of a loss-of-function-related genetic alteration in the PAPPA gene or its regulatory or promoter sequences in a blood sample obtained from the subject, wherein the presence of a genetic alteration is indicative of breast cancer.

The loss-of-function-related genetic alteration in the PAPPA gene or its regulatory or promoter sequences can be any genetic alteration/mutation, including one or more of a point mutation, deletion, loss of heterozygosity, translocation event, insertion, chromosomal breakage or methylation. Preferably the loss-of-function-related genetic alteration in the PAPPA gene or its regulatory or promoter sequences is hypermethylation or deletion.

In a tenth aspect, there is a method for screening for breast cancer in a subject, comprising detecting presence and/or level of PAPPA in a blood sample obtained from the subject, wherein the absence of PAPPA or PAPPA activity in the sample, or the presence of PAPPA, or PAPPA activity, at a reduced level compared to a control is indicative of breast cancer. Preferably “control” refers to PAPPA levels measured in a sample from a healthy individual.

The presence of PAPPA can be determined using any of the methods disclosed herein, including the use of a PAPPA-specific antibody or a probe for the PAPPA gene, mRNA or a specific PAPPA mutation.

### 5. Diagnosis

The present invention can also be used to aid primary diagnosis of breast cancer, as an adjunct to other conventional diagnostic techniques.

As used herein, the term “primary diagnosis” refers to the first characterisation of a tumour in a patient. Primary diagnosis is distinguished from diagnosis of secondary/metastatic cancers that are the result of spread from a primary tumour site, and also from the diagnosis of pre-invasive lesions, such as ductal carcinoma in-situ (DCIS).

As used herein, references to “aiding diagnosis” refers to aiding primary diagnosis, in the context of non-routine testing an individual to detect the presence of a disease, in particular breast cancer. In contrast to screening, in the context of diagnosis the patient is an individual who is presenting with symptoms of breast cancer or who has been motivated to seek medical attention due to symptoms of breast cancer. In certain embodiments, “aiding diagnosis” may also refer to stratification of a patient with a positive diagnosis of breast cancer, to further characterise the tumour.

As used herein, “symptoms of breast cancer” include, but are not limited to any of the following signs and symptoms:
A lump or thickening in or near the breast or in the underarm area.
A change in the size or shape of the breast.
A dimple or puckering in the skin of the breast.
A nipple turned inward into the breast.
Fluid, other than breast milk, from the nipple, especially if its bloody.
Scaly, red, or swollen skin on the breast, nipple, or areola (the dark area of skin around the nipple).
Dimples in the breast that looks like the skin of an orange.

A diagnosis of breast cancer can be made using various techniques, including:
Physical exam history: An exam of the body to check general signs of health, including checking for signs of disease, such as lumps or anything else that seems unusual. A history of the patient's health habits and past illnesses and treatments will also be taken.
Clinical breast exam (CBE): An exam of the breast by a doctor or other health professional. The doctor will carefully feel the breast and under the arms for lumps or anything else that seems unusual.
Mammogram: An x-ray of the breast, thermography, tomosynthesis or scintimammography.
Ultrasound: A procedure in which high-energy sound waves (ultrasound) are bounced off internal tissues or organs and make echoes. The echoes form a picture of body tissues called a sonogram. The picture can be printed to be looked at later.
MRI (magnetic resonance imaging): A procedure that uses a magnet, radio waves, and a computer to make a series of detailed pictures of areas inside the body. This procedure is also called nuclear magnetic resonance imaging (NMRI).
Blood chemistry studies: A procedure in which a blood sample is checked to measure the amounts of certain substances released into the blood by organs and tissues in the body. An unusual (higher or lower than normal) amount of substance can be a sign of disease in the organ or tissue that makes it.
Biopsy: The removal of cells or tissues so they can be viewed under a microscope by a pathologist to check for signs of cancer. If a lump in the breast is found, the doctor may need to remove a small piece of the lump. Four types of biopsies are as follows:
   Excisional biopsy: Removal of entire lump of tissue.
   Incisional biopsy: Removal of part of a lump or a sample of tissue.
   Core biopsy: Removal of tissue using a wide needle.
   Fine needle aspiration (FNA) biopsy: Removal of tissue or fluid, using a thin needle.
      The present invention can be used separately or in combination with any of the above diagnostic methods, to make a diagnosis.

According to the eleventh aspect, there is a method for aiding primary diagnosis of breast cancer in a patient, comprising detecting the presence of a loss-of-function-related genetic alteration in the PAPPA gene or its regulatory or promoter sequences in a sample obtained from the patient, wherein if a genetic alteration is present the result is indicative of breast cancer. The sample is selected from blood, tissue and nipple aspirates. The sample is preferably blood or nipple aspirates.

The loss-of-function-related genetic alteration in the PAPPA gene or its regulatory or promoter sequences can be any genetic alteration/mutation, including one or more of a point mutation, deletion, loss of heterozygosity, translocation event, insertion, chromosomal breakage or methylation. Preferably the loss-of-function-related genetic alteration in the PAPPA gene or its regulatory or promoter sequences is hypermethylation or deletion.

According to a further twelfth aspect, there is a method for aiding primary diagnosis of breast cancer in a patient, comprising detecting the presence and/or level of PAPPA, or PAPPA activity, in a sample obtained from the patient, wherein if PAPPA, or PAPPA activity, is not present, or is present at a reduced level compared to a control, the diagnosis is positive for breast cancer.

Preferably “control” refers to PAPPA levels measured in a sample from a healthy individual.

Again, the sample is selected from blood, tissue and nipple aspirates, and is preferably blood or nipple aspirates.

A further aspect of present invention provides a method for aiding primary diagnosis of breast cancer in a patient, comprising identifying the proportion of mitotic cells in a sample obtained from the patient that are in prophase or pro-metaphase and comparing to a pre-determined cut-off value, wherein if the proportion of cells in prophase or pro-metaphase is the same or greater than the cut-off value the diagnosis is positive for breast cancer. The sample is preferably nipple aspirates or breast tissue.

The cut-off value is at least 30% of the mitotic cells in the sample, wherein at least five of the cells in the sample are in mitosis.

Two or more of the above aspects may be carried out to aid the diagnosis, in conjunction with conventional diagnostic techniques.

The methods outlined above can be carried out using any of the techniques disclosed herein.

### 6. Prognostication of Disease Outcome

The methods of the present invention may also be used to predict disease progression in a patient who has been diagnosed with invasive breast cancer.

The invention provides a method for predicting disease progression in a patient who has been diagnosed with invasive breast cancer, comprising identifying the proportion of mitotic cells in a tissue sample or nipple aspirates obtained from the patient that are in prophase or prometaphase and comparing to a pre-determined cut-off value, wherein if the proportion of cells in prophase or prometaphase is the same or greater than the cut-off value, the prediction is reduced disease-free and overall survival.

In a related aspect of the invention, a method for predicting disease progression in a patient who has been diagnosed with invasive breast cancer comprises detecting the presence of a loss-of-function-related genetic alteration in the PAPPA gene or its regulatory or promoter sequences in a sample obtained from the patient, wherein the presence of a genetic alteration predicts reduced disease-free and overall survival. The loss-of-function-related genetic alteration in the PAPPA gene or its regulatory or promoter sequences is a one or more of a point mutation, deletion, insertion, translocation, chromosomal breakage, loss of heterozygosity or methylation, and preferably deletion of hypermethylation.

Another related aspect provides a method for predicting disease progression in a patient who has been diagnosed with invasive breast cancer, comprising detecting the presence and/or level of PAPPA in a biological sample obtained from the patient, wherein the absence of PAPPA, or its presence at a reduced level compared to a control, is predictive of reduced disease-free and overall survival.

The term “control” may refer to PAPPA levels measured in a sample from a healthy individual, or may refer to the patient's baseline PAPPA levels determined from a sample taken from the patient prior to or during treatment.

Preferably these methods are carried out using a liquid biopsy sample, such as blood or nipple aspirates.

The inventors have identified that one cause of PAPPA suppression in breast cancer cells (or pre-cancerous cells) is due to methylation of its DNA, primarily the PAPPA promoter region. DNA methylation, caused primarily by covalent addition of methyl groups to cytosine within CpG dinucleotides, occurs primarily in promoter regions of genes due to the large proportion of CpG islands found there. Hypermethylation results in transcriptional silencing.

Detecting the presence or absence of cancer by determining the methylation state of specific genes is known (but not in the context of PAPPA), and conventional methods for doing this may be adapted for use in the present invention. For example, methylation-specific PCR (MSP) has been used to determine the methylation status of specific genes. This technique, referred to also as MethyLight is described in Eads et al, Nucleic Acids Res. 2000; 28(8), and Widschwendler et al, Cancer Res., 2004; 64:3807-3813, the content of each of which is incorporated herein by reference. Alternative methods include Combined Bisulphate Restriction Analyses, Methylation-sensitive Single Nucleotide Primer Extension and the use of CpG island microarrays. Commercially available kits for the study of DNA methylation are available. Accordingly, the present invention makes use of conventional methods for determining the methylation state of the PAPPA gene or its regulatory promoter sequences.

MethyLight is a high-throughput quantitative methylation assay that utilises fluorescence-based real-time PCR (TaqMan®) technology that requires no further manipulations after the PCR step. MethyLight is a highly sensitive assay, capable of detecting methylated alleles in the presence of a 10,000-fold excess of unmethylated alleles. The assay is also highly quantitative and can very accurately determine the relative prevalence of a particular pattern of DNA methylation using very small amounts of template DNA.

MethyLight can be used to determine the methylation state of the PAPPA gene or regulatory or promoter regions in a sample of genomic DNA obtained for the patient's sample. Determination of the methylation state of the PAPPA gene may comprise the following steps:

i. Genomic DNA is extracted from the breast tissue sample and treated with sodium bisulfite to convert unmethylated cytosines to uracil residues (methylated residues are protected);
ii. Primers and probes designed specifically for bisulfite-modified DNA, such as those detailed in Table 2 in Example 1 are used to amplify the bisulfite-targeted DNA sample. The primer/probe sets used include a methylated set specific for the PAPPA gene (for example, see SEQ ID Nos. 7-9 in Table 2) and a set specific for a reference gene (COL2A1) (for example, see SEQ ID Nos. 31-33 in Table 2);
iii. The data is analysed and Ct values are calculated, for example by using ABI Step one plus software;
iv. The percentage of fully methylated PAPPA molecules at the specific locus is calculated by dividing the PAPPA:COL2A1 ratio of a sample by the PAPPA:COL2A1 ratio of a positive control sample (for example, Sssl treated HeLa genomic DNA) and multiplying by 100.

Since MethyLight reactions are specific to bisulfite converted DNA, the generation of false positive results is precluded.

Although DNA methylation (hypermethylation) is one cause of PAPPA suppression, there may be other causes. For example, the PAPPA gene (or its regulatory sequences) may be mutated leading to transcriptional silencing. Point mutations, deletions, loss of heterozygosity, translocations etc. may all cause the PAPPA gene to lose transcriptional activity. While modification at the genetic level may cause reduced (or no) expression of PAPPA, it may also be that modification (mutation) at the genetic level results in expression of PAPPA with reduced or no functional activity. Accordingly, the present invention envisages that PAPPA activity levels be used to help make a diagnosis. Mutation hot spots may be identified which contribute to the loss of activity and identifying such hot spots in a patient sample can also contribute to the diagnosis.

Loss of heterozygosity may contribute to PAPPA suppression in breast tissue. All cells contain two copies of somatic genes—one copy inherited from each parent. If a cell develops a mutation in one of the alleles of a tumour suppressor gene like PAPPA, loss of the remaining allele (termed “loss of heterozygosity” or “LOH”) can initiate tumourigenesis.

Loss of heterozygosity can be measured using various techniques, including the following: semi quantitative RT-PCR analysis. PAPPA is localised to human chromosome 9q32-33.1. Total RNA can be extracted using commercially available RNA extraction kits and reverse transcription can be performed using a reverse transcriptase enzyme. Unique primers can be designed within the PAPPA gene region and RT-PCR reactions can be performed in a thermal cycler. Levels of expression of PAPPA gene can be determined by the ratio of the band intensity of PAPPA gene compared to an endogenous control.

Real-time PCR reactions can also be performed to quantitatively confirm the results obtained from RT-PCR as will be appreciated by the skilled person. Unique primers can be designed for PAPPA and an endogenous control. Real-time PCR can be carried out to generate a standard curve for each gene under investigation. The fold reduction of PAPPA can be normalised to that of an endogenous control to compensate for the amount of RNA in each sample and also to account for the differences in the efficiency of the reverse transcription reaction.

Other methods used for the detection of loss of heterozygosity are Southern blotting, high-resolution PCR based fluorescence quantitation using capillary electrophoresis systems, amplification of microsatellites by PCR using radiolabelled nucleotides followed by autoradiography and next generation sequencing (Ion Torrent™, Life Technologies).

### Southern Blotting

Tumour and reference control DNA is isolated and digested with restriction enzymes. Digested DNA is subjected to Southern blot. ³²P-labelled DNA probes are designed to bind to restriction fragment length polymorphisms (RFLP). DNA probes are hybridised to the blot and then visualised by autoradiography. The advantage of Southern blotting is the frequency of false positives or negatives is lower than other techniques. The disadvantages are that this technique is low throughput and the amount of DNA required. If DNA is from FFPE tissues the fragmented nature of this DNA may inhibit the ability to be digested by restriction enzymes. Also, newer technologies described below have replaced radioactive labels with fluorescent labels.

### PCR-Based Microsatellite Analysis

Primers are designed to amplify the microsatellite loci. For example, suitable primers for the amplification of KLF4 gene mapping within the FRA9E CFS regions are: 5′ CAGAGGAGCCCAAGCCAAAGAG 3′ (SEQ ID NO. 43) and 5′ CACAGCCGTCCCAGTCACAGT 3′ (SEQ ID NO. 44). One primer from each pair is labelled with T4 kinase and γ [³²P]ATP. PCR amplification is performed using DNA, labelled primer and unlabelled primer. PCR samples are separated by formamide and urea polyacrylamide gel electrophoresis. After fixing and drying, the gel is exposed to X-ray film. Bands are analysed by laser densitometry and the imbalance is defined as the ratio of allele intensities in the tumour sample relative to the ratio of alleles in normal DNA. The advantage of this technique is that it can analyse DNA from fresh tissue, FFPE tissue, and cells. However, DNA from FFPE tissue may be degraded and therefore too short to be amplified by PCR. The disadvantage of this technique is the potential for false positives due to mis-priming. The technique is labour intensive and requires radioactive labels. This technique is usually carried out together with QRT-PCR PAPPA is one of the 16 genes found in this site.

### Fluorescence In Situ Hybridisation (FISH)

Samples from FFPE, frozen tissue sections, or cells are fixed and permeabilised. Oligonucleotide pairs are hybridised to target RNA. The use of multiple fluorescent-labelled probes enables multiplexing of targets. Samples are viewed under a fluorescence microscope. Alternatively, to detect DNA a metaphase spread from cells is generated and repetitive DNA sequences are blocked. A probe is designed to hybridise to the target and tagged with fluorophores, targets for antibodies, or with biotin. Samples are viewed under a fluorescence microscope. The advantage of FISH is that it detects balanced translocations. Cells are individually analysed and therefore a heterogeneous population or contaminating normal cells would not affect the analysis. The disadvantages of FISH are that it is labour intensive and in order to target DNA, and metaphase spreads need to be generated from cells.

### Comparative Genomic Hybridisation (CGH)

CGH technology can detect DNA copy number changes across the entire genome by comparing the hybridisation intensity of the subject samples compared to a reference sample. The DNA is fluorescently labelled and mixed with unlabelled human cot-1 DNA to supress repetitive sequences. The DNA mix is hybridised to a normal metaphase spread. The ratio of subject and reference binding is determined by epifluorescence microscopy. The resolution is quite low; to detect a single copy loss the region must be at least 5-10 Mb and amplification changes less than 1 Mb are undetectable. There have been advances to improve the resolution by using array CGH (aCGH) instead of metaphase spreads. The aCGH uses similar technology to microarrays. High resolution aCGH are able to detect structural variations of a resolution of 200 bp. However, aCGH has proved difficult to use with DNA from FFPE. Also, aCGH can require large quantities of subject DNA.

### SNP Microarray

Various SNP microarray technologies are available commercially, e.g. from Affymetrix and Illumina, and can be used in the present invention. The Affymetrix technology uses the same basic principles as DNA microarray: solid surface DNA capture, DNA hybridisation and fluorescence microscopy. The Illumina platform uses oligonucleotide bound BeadArray in micro-wells on either fibre optic bundles or planar silica slides. Normal and tumour DNA is fluorescently labelled and bound to predesigned SNP arrays. For each SNP, four to six probes are used. The fluorescent intensity of DNA bound to perfect match probes against mismatch probes is analysed. SNP microarray platforms can be utilised for high-throughput LOH detection. Microarrays can be used for DNA or RNA-analysis applications. The accuracy of SNP calls are based upon the model used to analyse the data including the different definitions of LOH thresholds. The frequently used Hidden Markov Model-based approaches (Green et al., BMC Cancer; 2010, 10:195) allow detection of LOH from hemizygous deletion of single alleles, but cannot detect copy-number neutral LOH.

### Next Generation Sequencing

Next generation sequencing technology is moving to replace SNP-arrays for the detection of LOH. Whole genome sequencing, targeted resequencing of a gene of interest, or exome sequencing of a gene of interest can be applied to detect SNPs. A range of tumour DNA, or tumour DNA compared to reference DNA can be sequenced by synthesis or by ligation technology. The DNA sequence is read by fluorescence or a change is pH, depending on the instrument used. The advantages of next generation sequencing are that it is high throughput, fast and has a low cost per base. The disadvantages are that the whole gene of interest may not be covered due to repetitive sequences or high GC content. The accuracy of SNP calls are based upon the model used to analyse the data including the different definitions of LOH thresholds.

As mentioned previously, point mutations may be responsible for PAPPA loss or PAPPA loss-of-functional activity. Point mutations are genetic changes where the mutation of a single DNA base to another base can lead to a non-functional protein. These point mutations can be further classified into nonsense mutations, where the mutant gene brings the protein synthesis to a premature halt; missense mutations, where the altered codon results in the insertion of an incorrect amino acid into the protein; and frame-shift mutations, where the loss or gain of one or more nucleotides causes the codons to be misread, resulting in non-functional proteins. There are a variety of methods available for the detection of point mutations in molecular diagnostics. The choice of the method to be used depends on the specimen being analysed, how reliable the method is, whether the mutations to be detected are known before analysis and the ratio between wild-type and mutant alleles.

Denaturing gradient gel electrophoresis is a further technique for mutation detection, particularly for point mutations. A prolonged (48 hr) proteinase K digestion method or DNA easy kit (Qiagen) can be used to extract genomic DNA. Double stranded DNA (PCR fragments of 1 kb) can be generated by multiplex PCR reaction covering the whole of the PAPPA coding region. In order to increase the efficiency of detection GC clamps can be attached to one of the PCR primers. The DNA can then be subjected to increasing concentrations of a denaturing agent like urea or formamide in a gel electrophoresis set up. With increasing concentrations of denaturing agent domains in the DNA will dissociate according to their melting temperature (Tm). DNA hybrids of 1 kb usually contain about 3-4 domains, each of which would melt at a distinct temperature. Dissociation of strands in such domains results in the decrease of electrophoretic mobility, and a 1 bp difference is sufficient to change the Tm. Base mismatches in the heteroduplices lead to a significant destabilisation of domains resulting in differences in Tm between homoduplex and heteroduplex molecules. The homo and heteroduplices will be detected by silver staining after gel electrophoresis. This method offers the advantage that 100% of point mutations can be detected when heteroduplices are generated from sense and antisense strands (Cotton R G, Current methods of mutation detection, Mutat Res 1993; 285: 125-44).

Alternative methods available for the detection of point mutations include PCR-single stranded conformation polymorphism, heteroduplex analysis, protein truncation test, RNASE A cleavage method, chemical/enzyme mismatch cleavage, allele specific oligonucleotide hybridisation on DNA chips, allele specific PCR with a blocking reagent (to suppress amplification of wild-type allele) followed by real time PCR, direct sequencing of PCR products, pyrosequencing and next generation sequencing systems.

As mentioned previously, PAPPA loss and/or PAPPA loss-of-functional activity may be due to insertions, deletions, translocation, loss of heterozygosity, chromosomal breakage and frame-shift mutations. The technique of pyrosequencing can be used for detection of insertions, deletions, frame-shift mutations. Pyrosequencing is based on the sequencing-by-synthesis principle. In this method a single-stranded PCR/RT-PCR fragment is used as a template for the reaction. During the process of DNA replication after nucleotide incorporation, released PPi (inorganic phosphate) is converted to light by an enzymatic cascade; ATP sulfurylase which converts PPi to ATP in the presence of APS. This ATP would further drive the luciferase mediated conversion of luciferin to oxyluciferin that generates visible light, which can be detected by a CCD sensor and is visible as a peak in the pyrogram (Ronaghi, M., Uhlen, M., and Nyren, P, A sequencing method based on real-time pyrophosphate, Science; 1998b 281: 363-365). The light signal generated is linearly proportional to the nucleotides incorporated.

A prolonged (48 hr) proteinase K digestion method or DNA easy kit (Qiagen) can be used to extract genomic DNA from the patient sample. PCR and sequencing primers for the PAPPA gene can be designed for use in pyrosequencing. PCR products can be bound to streptavidin-sepharose, purified washed and denatured using NaoH solution and washed again. Then the pyrosequencing primer can be annealed to the single-stranded PCR product and the reaction carried out on, for example, a Pyromark ID system (Qiagen) according to the manufacturer's instructions.

Other methods available for detecting insertions/deletions and frame-shift mutations are big dye terminator sequencing, next generation sequencing systems and heteroduplex analysis using capillary/microchip based electrophoresis.

Alternative methods of the invention require determining the presence (or absence) or the level of PAPPA in a patient sample. This can be carried out by determining protein levels, or by studying the expression level of the gene coding for the protein. As used herein the term “expression level” refers to the amount of the specified protein (or mRNA coding for the protein) in the breast tissue sample. The expression level is then compared to that of a control. The control may be a sample of a person that is known to not have cancer or may be a reference value. It will be apparent to the skilled person that comparing expression levels of a control and the test sample will allow a decision to be made as to whether the expression level in the test sample and control are similar or different.

Methods of measuring the level of expression of a protein from a biological sample are well known in the art and any suitable method may be used. Protein or nucleic acid from the sample may be analysed to determine the expression level, and examples of suitable methods include semi-quantitative methods such as in situ hybridisation (ISH) fluorescence and in situ hybridisation (FISH), and variants of these methods for detecting mRNA levels in tissue or cell preparations, Northern blotting, and quantitative PCR reactions. The use of Northern blotting techniques or quantitative PCR to detect gene expression levels is well known in the art. Kits for quantitative PCR-based gene expression analysis are commercially available, for example the Quantitect system manufactured by Qiagen. Simultaneous analysis of expression levels in multiple samples using a hybridisation-based nucleic acid array system is well known in the art and is also within the scope of the invention. Mutation-specific PCR may also be used, as will be appreciated by the skilled person.

PAPPA levels in a sample can be determined using conventional immunological detection techniques, using conventional anti-PAPPA antibodies. The antibody having specificity for PAPPA, or a secondary antibody that binds to such an antibody, can be detectably-labelled. Suitable labels include, without limitation, radionuclides (e.g. ¹²⁵I, ¹³¹I, ³⁵S, ³H, ³²P or ¹⁴C), fluorophores (e.g. Fluorescein, FITC or rhodamine), luminescent moieties (e.g. Qdot nanoparticles supplied by Quantum Dot Corporation, Palo Alto Calif.) or enzymes (e.g. alkaline phosphatase or horse radish peroxidase).

Immunological assays for detecting PAPPA can be performed in a variety of assay formats, including sandwich assays e.g. (ELISA), competition assays (competitive RIA), bridge immunoassays, immunohistochemistry (IHC) and immunocytochemistry (ICC). Methods for detecting PAPPA include contacting a patient sample with an antibody that binds to PAPPA and detecting binding. An antibody having specificity for PAPPA can be immobilised on a support material using conventional methods. Binding of PAPPA to the antibody on the support can be detected using surface plasmon resonance (Biacore Int, Sweden). Anti-PAPPA antibodies are available commercially (e.g. HPA001667 from Sigma-Aldrich, MA1-46425 (5H9) from Thermo Scientific, OASA03208 from Aviva Systems Biology and AO230 from Dako). The immuno-detection of PAPPA is also disclosed in U.S. Pat. No. 6,172,198, the content of which incorporated herein by reference.

In order to enable immunohistochemical detection of PAPPA within a breast tissue sample, formalin-fixed, paraffin-embedded breast tissue sections are prepared and mounted on SuperFrost++ charged slides. Following epitope retrieval by proteolytic digestion, endogenous peroxidase activity is quenched and the sections are incubated with a first anti-PAPPA antibody (available, for example, from DAKO). The section is then further incubated with a polymer-linked secondary antibody and peroxidase which enables a chromogenic signal to develop following addition with DAB, thereby allowing binding of the first antibody to the PAPPA protein to be detected visually. The immunohistochemical procedure described above can be fully automated using commercially available immunostainers.

PAPPA protein expression can be classified using conventional methods, for example, membrane and cytoplasmic staining intensity can be evaluated using the following scoring system: negative (0), no staining is observed; weakly positive (1+), a faint/barely perceptible membrane/cytoplasmic staining is detected in more than 25% of cells; moderately positive (2+), weak staining is detected in more than 25% of cells; strongly positive (3+), strong membrane/cytoplasmic staining is detected in more than 25% of cells. Any focal staining of less than 25% of tumour cells is considered as 1+.

For analysis of a relatively small number of PAPPA proteins, a quantitative immunoassay such as a Western blot or ELISA can be used to detect the amount of protein (and therefore level of expression) in a breast tissue sample. Semi-quantitative methods such as immunohistochemistry (IHC) and immunocytochemistry (ICC) can also be used.

To analyse a larger number of samples simultaneously, a protein array may be used. Protein arrays are well known in the art and function in a similar way to nucleic acid arrays, primarily using known immobilised proteins (probes) to “capture” a protein of interest. A protein array contains a plurality of immobilised probe proteins. The array contains probe proteins with affinity for PAPPA.

Alternatively, 2D Gel Electrophoresis can be used to analyse simultaneously the expression level of PAPPA. This method is well known in the art: a sample containing a large number of proteins are typically separated in a first dimension by isoelectric focusing and in a second dimension by size. Each protein resides at a unique location (a “spot”) on the resulting gel. The amount of protein in each spot, and therefore the level of expression, can be determined using a number of techniques. An example of a suitable technique is silver-staining the gel followed by scanning with a Bio-rad FX scanner and computer aided analysis using MELANIE 3.0 software (GeneBio). Alternatively, Difference Gel Electrophoresis (DIGE) may be used to quantify the expression level (see Von Eggeling et al; Int. J. Mol Med. 2001 October; 8(4):373-7.

Typically, the presence of PAPPA or PAPPA activity is considered to be at a reduced level compared to a control if PAPPA is not present or is present at a level less than 90%, 80%, 70%, 60%, 50%, 40%, 30% or 20% compared to the control. Most typically, if the presence of PAPPA or PAPPA activity is considered to be at a reduced level compared to a control, PAPPA will be present at an amount between 40%-80% of that of the control. Most typically, PAPPA will be present at a level between 50%-70%, e.g. approx. 60% compared to that of a control. The control can be a patient sample from normal breast tissue or fluid biopsy, or may be a reference value.

The methods according to certain aspects of the present invention are carried out typically to establish whether PAPPA is present in the tissue or liquid biopsy sample at reduced levels compared to a control. It is also envisaged that PAPPA protein may be present at or near to normal levels, but the expressed protein is inactive, or active at reduced levels. Accordingly, the invention encompasses monitoring the activity of PAPPA. Therefore, all references herein to determining whether PAPPA is present (or is present at a reduced level) compared to a control, encompass monitoring the functional activity of PAPPA.

PAPPA activity can be measured using conventional techniques. For example, PAPPA activity can be determined by examining IGFBP-4 proteolytic activity in a sample. Methods for detecting PAPPA activity are disclosed in US patent publication No. 2005/0272034, the content of which is incorporated herein by reference. Alternatively, loss of PAPPA activity may also be determined by mutation-specific PCR analysis.

In one embodiment, PAPPA activity may be detected by screening for proteolytic cleavage of its substrate IGFBP-4 using immunoblotting. PAPPA secreted into the medium can be detected by incubating the media samples in a buffer, such as 50 mM Tris (pH 7.5) supplemented with IGFBP-4. Samples can then be incubated (for example, at 37° C. for 4 hrs) and the proteolytic products detected by immunoblotting using available commercial antibodies against IGBP4 protein.

Alternatively, PAPPA activity can be detected by using an ELISA (Enzyme linked immunosorbent assay), wherein specific antibodies against PAPPA are immobilised in the well of a microtitre plate. After washing away unbound protein the activity of PAPPA can be measured using a synthetic substrate which liberates a coloured product only if the primary specific reaction between PAPPA and its antibody has occurred and the bound PAPPA is active. The colour developed is quantified spectrophotometrically using a microplate reader.

Alternatively, the interaction between PAPPA and its substrate IGFBP-4 can also be assessed using Biacore (Surface plasmon resonance technology) or Fluorescence polarisation assay. These methods offer the advantage of being very sensitive and specific and can easily be adapted to develop a high-throughput assay.

As a control for the above described methods a mutant PAPPA protein (E483Q) which is proteolytically inactive may be used.

PAPPA activity may be reduced by greater than 20%, 30%, 40%, 50%, 60%, 70%, 80%, 90% or greater than 90% compared to a control, in a tissue or liquid biopsy sample from a patient.

Other aspects of the invention are directed to determining whether cells in a breast tissue sample (or nipple aspirates) are stalled in mitosis. The present invention provides an in vitro method for determining whether breast cells are stalled in mitosis, by identifying a delayed mitotic phenotype. Identification of this phenotype comprises identifying the proportion of mitotic cells in a tissue sample obtained from a patient that are in prophase or prometaphase and comparing to a pre-determined cut-off value.

The pre-determined cut-off value is at least 30% and preferably at least 33%. Therefore, if at least 30% of mitotic cells in the tissue sample are identified as being in prophase or pro-metaphase then the delayed mitotic phenotype is identified in the tissue sample. The pre-determined cut-off value may also be set higher than this, for example, at least 35%, 40%, 50%, 60%, 70% or more.

In order for the analysis to be statistically significant, at least five of the cells within the tissue sample must be undergoing mitosis. If at least 30% of these at least five mitotic cells are identified as being in prophase or prometaphase then the tissue sample is identified as having a delayed mitotic phenotype. For example, if five of the cells within the tissue sample are in identified as being in mitosis, at least two of these cells must be in prophase/prometaphase in order for the mitotic delay phenotype to be identified.

More preferably the proportion of mitotic cells in prophase/prometaphase in a breast tissue (or nipple aspirates) sample from a patient having the delayed mitotic phenotype is greater than 33%, 35%, 40%, 45%, 50%, 55%, 60%, 65%. Typically, a “control” value for non-invasive healthy breast tissue cells undergoing mitosis would be approximately 10-25%, e.g. 23% cells in prophase/prometaphase.

If fewer than five cells in a tissue sample are undergoing mitosis then the analysis of the proportion of mitotic cells that are in prophase/prometaphase will not be sufficiently significant to enable the delayed mitotic phenotype to be identified according to the methods of the invention. Therefore, the methods require there to be at least five mitotic cells in the tissue sample being analysed at the time of analysis.

Detection of whether the cells of the tissue sample (or nipple aspirates) are in prophase or pro-metaphase can be carried out using techniques conventional in the art. For example, immuno-detection techniques using specific antibodies are often used to characterise the mitotic phase of a cell. Immunohistochemistry (IHC) is an immuno-detection technique and refers to the process of detecting antigens in cells of a tissue section by visualising an antibody-antigen interaction. This can be achieved by tagging an antibody with a reporter moiety, preferably a visual reporter such as a fluorophore (termed “immunofluorescence”) or by conjugating an antibody to an enzyme, such as peroxidase, that can catalyse a colour-producing reaction that can be detected and observed.

H3S10 phosphorylation (H3S10ph) is a mitosis-specific modification essential for the onset of mitosis; the phosphorylation of the serine 10 at Histone H3 is important for chromosome condensation. Antibodies specific for H3S10ph are commercially available (e.g. Millipore and Active Motif) as are kits for carrying out mitotic assays.

Alternative markers of mitosis, such as phosphor-specific nuclear-laminin are also available commercially and may be utilised in the methods of the invention. Other methods for assessing and characterising the different phases of mitosis such as the use of specialist stains and visual morphological analysis are well known in the art as will be appreciated by the skilled person.

The immuno-assay analysis can be carried out to provide a “snapshot” of the different phases of mitosis for a tissue sample. In this way, a mitotic phase distribution analysis is obtained which is then used to characterise the proportion of mitotic cells that are in prophase or prometaphase.

In order to enable immunohistochemical detection of a mitotic marker (such as H3S10ph), formalin-fixed, paraffin-embedded breast tissue sections are prepared and mounted on SuperFrost++ charged slides. Following heat-mediated epitope retrieval, endogenous peroxidase activity is quenched and the sections are incubated with a first antibody (suitable H3S10ph antibodies are available, for example, from Millipore) which specifically recognises mitotic markers (such as phosphorylated H3S10) within mitotic cells. The section is then further incubated with a polymer-linked secondary antibody and peroxidase which enables a chromogenic signal to develop following addition with DAB, thereby allowing binding of the first antibody to the mitotic marker to be detected visually. The immunohistochemical procedure described above can be fully automated using commercially available immunostainers.

In order to analyse mitotic phase distribution in a breast tissue sample or other patient sample (e.g. nipple aspirate) or cultured cell line, at least two consecutive serial sections from each sample and at least two cytospin preparations for each cell line or body fluid (e.g. aspirate) are immunolabelled as described above and five to twenty high power fields (400× magnification) are image captured and a minimum of 5 mitotic cells for each sample are used to determine the mitotic phase distribution. All mitotic cells within the captured fields can be classified based on their chromosomal morphology as prophase/prometaphase, metaphase, anaphase and telophase, according to classical morphological criteria. A population of cells is classified as ‘delayed’ if at least 30% of mitotic cells reside in prophase/prometaphase.

The breast tissue sample (or nipple aspirates) analysed by any of the methods described herein may be taken from a patient exhibiting proliferative lesions. The tissue sample may include pre-invasive lesions, including ductal carcinoma-in-situ (DCIS), lobular carcinoma-in-situ (LCIS) and Paget's disease of the nipple, and proliferative lesions with uncertain malignant potential, including such entities as lobular neoplasia, lobular intraepithelial neoplasia, atypical lobular hyperplasia (ALH), flat epithelial atypia (FEA), atypical ductal hyperplasia (ADH) microinvasive carcinoma, intraductal papillary neoplasms and phyllodes tumour. Methods for taking a sample from a patient (biopsy) will be apparent to the skilled person.

The content of all publications referred to herein is incorporated by reference. The invention is described with reference to the accompanying drawings, by the following non-limiting examples.

### Example 1

### Mitotic Delay Phenotype and PAPPA Expression Levels as Markers of Breast Cancer

### Methods

### Tissue Specimens

Formalin-fixed, paraffin-embedded tissue was retrieved from the archives of the Department of Pathology at UCL (UCL Hospitals, London, UK) and included: invasive breast cancer (n=182, 156 of which were included for analysis of mitotic phase distribution); ductal carcinoma in situ (DCIS; n=81, 69 evaluable); normal breast tissue from reduction mammoplasty specimens (n=33, evaluability not relevant); normal breast tissue from pregnant patients (n=5, all evaluable); colon adenocarcinoma (n=41, all evaluable); transitional cell carcinoma of the bladder (n=27, all evaluable); penile squamous cell carcinoma (n=33, all evaluable); gastric adenocarcinoma (n=21, all evaluable); malignant melanoma (n=21, all evaluable); small cell lung cancer (n=30, all evaluable); and non-Hodgkin lymphoma (n=29, all evaluable). Cases were selected on the basis of available histological material and clinico-pathological information. Histological specimens had been reviewed by a qualified pathologist at diagnosis and assessed for histological subtype and nuclear grade according to the World Health Organization (WHO) criteria. A case was evaluable for mitotic phase distribution analysis if at least five mitotic cells were found on the specimen. Ethical approval was obtained from the Joint UCL/UCLH Committees on the Ethics of Human Research.

### Cytospin Preparations

To prepare cell monolayers, 0.5×10⁶ cells were cytospun onto glass slides at 800×g for 5 min using a Cytospin 4 cytocentrifuge (Thermo Scientific), air dried and fixed in 4% neutral buffered formalin overnight.

### Antibodies

PAPPA rabbit polyclonal antibody (PAPPA PAb) was raised against a synthetic peptide (aa1384-1399) following a 28-day immunisation protocol (Eurogentec). Other antibodies used include Histone H3 phosphorylated on serine 10 (H3S10ph) from Millipore (06-570), PAPPA from DAKO (A0230), β-actin (AC-15) from Sigma, CD29 (HUTS-21) from BD Pharmingen, β1-integrin (MAB1959) from Chemicon and Alexa Fluor 594 from Invitrogen.

### Immunohistochemistry

Section deparaffinisation, antigen retrieval and immuno-staining were performed using the Bond III Autostainer and Bond Polymer Refine Detection kit (Leica) according to the manufacturer's instructions. Heat-mediated antigen retrieval and proteolytic digestion were used for H3S10ph and PAPPA antigens, respectively. Primary antibodies were applied for 40 min at the following dilutions: PAPPA at 1/200; H3S10ph at 1/4000. Cytospin preparations were immuno-stained using the same protocol without the deparaffinisation step. Incubation without primary antibody was used as a negative control and sections of tonsil and placenta were used as positive controls for H3S10ph and PAPPA antibodies, respectively.

### Mitotic Phase Distribution Analysis

Two consecutive serial sections from each tissue sample and two cytospin preparations for each cell line were immuno-stained for H3S10ph for analysis of mitotic phase distribution. A minimum of 5 mitotic cells from 5-20 fields at 400× magnification were captured with a CV12 CCD camera and image capturing software (SIS). Cases were excluded from analysis if less than 5 mitotic cells were found on the specimen. Mitotic cells were classified as prophase/prometaphase, metaphase, anaphase or telophase according to conventional morphological criteria.

### Tissue Dissection

Tissue sections were deparaffinised, stained with Mayer's haematoxylin for 5 seconds, and air dried. A 100× magnification field was needle micro-dissected and genomic DNA extracted following incubation in 55 μl of 1 mg/ml proteinase K (Sigma-Aldrich) at 55° C. for 48 h.

### DNA Methylation Analysis

Genomic DNA from cell lines and cases of invasive breast cancer (n=182), DCIS (n=81) and normal breast (n=34) were used for MethyLight analysis (Widschwendter, M. et al. Cancer research (2004) 64, 3807-3813). Nine cases of invasive breast cancer, six cases of DCIS and four cases of normal breast were excluded from analysis because insufficient material for DNA extraction was found on the specimen. DNA concentration was determined by NanoDrop spectrophotometry and DNA quality was verified using qPCR with a reference gene. Mean genomic amplification of all breast samples was calculated at 34.39 cycles (SD=2.07). For all samples 400 ng of genomic DNA was bisulfite-modified using the EZ DNA Methylation-Gold Kit (Zymo Research) according to the manufacturer's instructions. Unmodified Sssl treated genomic DNA (New England Biolabs) was used as positive control. Bisulfite-modified DNA was stored at −80° C. until use. Quantitative PCR analysis using MethyLight was performed for all samples. Nucleotide sequences for MethyLight primers and probes were designed in the promoter or 5′-end region of the gene of interest. Each MethyLight reaction at a specific locus covered on average 5-10 CpG dinucleotides. A detailed list of primer and probes (Metabion) for all analysed loci is provided in Tables 1 and 2. Two sets of primers and probes, designed specifically for bisulfite-modified DNA, were used for each locus; a methylated set for the gene of interest and a reference gene (COL2A1) to normalize for input DNA. Specificity of the reactions for methylated DNA was confirmed separately using Sssl treated human white blood cell DNA (heavily methylated). The percentage of fully methylated molecules at a specific locus (PMR, percentage methylated reference) was calculated by dividing the GENE OF INTEREST:COL2A1 ratio of a sample by the GENE OF INTEREST:COL2A1 ratio of the Sssl-treated human white blood cell DNA and multiplied by 100.

### Cell Culture

BT549, T47D, BT474, MDAMB157, MDAMB453, MCF10A and human mammary epithelial cells (HMEpC) were cultured as described in Rodriguez-Acebes et al, Am. J. Pathol, 2010; 177:2034-2045. HeLa Kyoto cells were cultured in DMEM (Invitrogen) supplemented with 10% FCS (Invitrogen) at 37° C. with 5% CO₂.

### Cell Synchronization

HeLa Kyoto cells were synchronised in S phase by a double thymidine block (Sigma). Briefly, a final concentration of 3 mM thymidine was added to the culture medium for 18 h, followed by release into fresh culture medium for 9 h and a second block with 3 mM thymidine for 17 h. HeLa Kyoto cells were synchronised in M phase by treatment with the Plk1 inhibitor BI2536 (SelleckChem) at final concentration of 5 ng/ml for 24 hours. Cell synchronisation was confirmed by flow cytometry.

### Cell Population Growth Assessment and Cell Cycle Analysis

Cell proliferation assessment and flow cytometric cell cycle analysis were performed as described in Rodriguez-Acebes et al, Am. J. Pathol, 2010; 177:2034-2045.

### RNA Interference

PAPPA was silenced by RNAi with a specific RNA duplex targeting PAPPA mRNA (Ambion s10042; sense 5′-GAGCCUACUUGGAUGUUAAtt-3′ (SEQ ID NO. 37) and antisense 5′-UUAACAUCCAAGUAGGCUCtg-3′ (SEQ ID NO. 38) or, alternatively, a pool of duplexes (ON-TARGETplus SMARTpool, Dharmacon). Non-targeting siRNA (Stealth RNAi Negative Control Med GC, Invitrogen) was used as negative control. All transfections were performed with Lipofectamine 2000 (Invitrogen). PAPPA siRNA at 100 nM final concentration was used to achieve efficient knock-down. Cells were harvested at the indicated time points post-transfection. Knock-down efficiency was assessed by qRT-PCR and/or Western blot.

### Real-Time PCR

Total RNA was isolated from cells and qRT-PCR was performed using 100 ng of total RNA as described in Tudzarova et al, EMBO J., 2010; 29:3381-3394. Primer sequences were: PAPPA forward 5′-ACAGGCTACGTGCTCCAGAT-3′ (SEQ ID NO. 39) and reverse 5′-CTCACAGGCCACCTGCTTAT-3′ SEQ ID NO. 40); RPLPO (ribosomal protein used as invariant control) forward 5′-CCTCATATCCGGGGGAATGTG-3′ SEQ ID NO. 41) and reverse 5′-GCAGCAGCTGGCACCTTATTG-3′ (SEQ ID NO. 42).

### Immunofluorescence

For immunofluorescence, HeLa Kyoto cells were grown on coverslips (12 mm #1 VWR International) and synchronised as described above. The synchronised cells were rinsed in PBS, fixed in 1% PFA, permeabilised with 0.1% Triton X-100/0.02% SDS, and blocked in 2% BSA. After the blocking step, phosphohistone H3 (H3S10ph) antibody was applied for 1 h at a dilution of 1/500 and after three washes with PBS, Alexa Fluor 594 antibody was added at a dilution of 1/300 for 1 h. Coverslips were washed three times in PBS and mounted with Vectashield non-fade DAPI (Vector Laboratories).

### Cell Population Growth Assessment and Cell Cycle Analysis

Cell proliferation assessment and flow cytometric cell cycle analysis were performed as described in Rodriguez-Acebes supra.

TABLE 1
Location of CpG islands for MitoCheck candidate genes

| | | | CpG island | CGs in CpG | Obs CpG/ | GC |
|---|---|---|---|---|---|---|
| Gene^{a} | Gene locus | CpG island location^{b} | length (bp) | island (no.) | Exp CpG | (%) |
| PLK1 | chr16: 23,590,521-23,616,371 (16p12.1) | 23597103-23598423 | 1320 | 88 | 0.768 | 60.6 |
| TPX2 | chr20: 29,751,378-29,892,452 (20q11.2) | 29790413-29791269 | 856 | 48 | 0.823 | 55.0 |
| KIF111 | chr10: 94,327,431-94,420,670 (10q23.3) | 94341250-94341728 | 478 | 38 | 1.052 | 55.0 |
| KIF112 | chr10: 94,327,431-94,420,670 (10q23.3) | 94341912-94343451 | 1539 | 89 | 0.793 | 54.3 |
| PAPP-A | chr9: 117,893,759-118,266,552 (9q33.1) | 117955868-117957241 | 1373 | 115 | 0.852 | 64.5 |
| SGOL1 | chr3: 20,177,087-20,202,687 (3p24.3) | 20176596-20177594 | 998 | 65 | 0.876 | 55.0 |
| PSMD8 | chr19: 43,557,030-43,566,304 (19q13.2) | 43556706-43558018 | 1312 | 81 | 0.747 | 59.4 |
| TUBB2C | chr9: 139,255,532-139,257,980 (9q34) | 139254197-139256673 | 2476 | 225 | 0.817 | 67.3 |

^{a}Human Genome Organisation gene name
^{b}DNA sequences sourced using genome.ucsc.edu, CpG island locations identified using cpgislands.usc.edu

TABLE 2
Primers and probes used for MethyLight reactions

| | Forward | Reverse | |
|---|---|---|---|
| Gene^{a} | primer sequence 5′-3′ | primer sequence 5′-3′ | Probe sequence 5′-3′ |
| KIF11 | CGAGCGTTGTATGTTGGGATT | CGCAACGAACGATAACATCTCA | 6-FAM-AACTACGCAAACATCCGCCG-BHQ-1 |
| | SEQ ID NO. 1 | SEQ ID NO. 2 | SEQ ID NO. 3 |
| PAPPA (I) | GCGTCGAGGTTTTTAAAGTTG | CCCAACTCCAAAACCGCATAT | 6-FAM-CCCTACACCGCCACCCGAA-BHQ-1 |
| | GTA SEQ ID NO. 4 | SEQ ID NO. 5 | SEQ ID NO. 6 |
| PAPPA (II) | GCGTGTTTGTGCGAGAGTTGT | CGCCTTCCGAATATACCCATT | 6-FAM-TCGCCCGAATATCTCTACGCCG |
| | SEQ ID NO. 7 | SEQ ID NO. 8 | CT-BHQ-1 SEQ ID NO. 9 |
| PLK1 (I) | GTTCGGGCGTTCGTGTTAAT | GCCGCGCAACACCATAA | 6-FAM-CCCTACGCAACAACAACCTTTAA |
| | SEQ ID NO. 10 | SEQ ID NO. 11 | ACCCG-BHQ-1 SEQ ID NO. 12 |
| PLK1 (II) | GGTTTTATCGGCGAAAGAGAT | CGACCCCGCACATAACG | 6-FAM-CCGACTACGTAAATCCACTAAAA |
| | TT SEQ ID NO. 13 | SEQ ID NO. 14 | CCT-BHQ-1 SEQ ID NO. 15 |
| PLK1 (II) | CGCGTTTCGTTGTTAGATTTG | AAAAACCCGCGCCCTAACTA | 6-FAM-CTTCCCACGACTCACCTAACCTC |
| | AG SEQ ID NO. 16 | SEQ ID NO. 17 | G-BHQ-1 SEQ ID NO. 18 |
| TPX2 | TGGCGATAGGATTTTGTTGTG | GACAACCTCCCGCAACTCTTT | 6-FAM-TTTCCTCCGTTTCCCGAACGAA- |
| | A SEQ ID No. 19 | SEQ ID NO. 20 | BHQ-1 SEQ ID NO. 21 |
| TUBB2C | AACGAACGCGCAAAATACTAC | CGTTTTGGTAGTTTATTTCGAG | 6-FAM-CCGACGCTCCGCCAACGCTTA-BHQ-1 |
| | A SEQ ID NO. 22 | ATTAGC SEQ ID NO. 23 | SEQ ID NO. 24 |
| SGOL1 | CGCACATTCGCTCAAATCC | CGTCGAGATTCGATCGTAGGTT | 6-FAM-CATCCGAACATCCACCTAACCCTA |
| | SEQ ID NO. 25 | SEQ ID NO. 26 | AAACG-BHQ-1 SEQ ID NO. 27 |
| PSMD8 | TTCGTCGGGTAAGCGTTTGTA | GCGACCGCCATCTTACGTAA | 6-FAM-CGGCGTTGTCGTAAATTAGGCGGT |
| | SEQ ID NO. 28 | SEQ ID NO. 29 | TT-BHQ-1 SEQ ID NO. 30 |
| COL2A1 (mod) | TCTAACAATTATAAACTCCAA | GGGAAGATGGGATAGAAGGGAA | 6-FAM-CCTTCATTCTAACCCAATACCTATCCC |
| | CCACCAA SEQ ID NO. 31 | TAT SEQ ID NO. 32 | ACCTCTAAA-BHQ-1 SEQ ID NO. 33 |
| COL2A1 (gen) | TCCGTAAGTGCAGCTTCTTTG | TGGAGCCCACAACTGTCAGA | 6-FAM-CAAAGTACAGAGTCAAGAGTTCCAAAG |
| | SEQ ID NO. 34 | SEQ ID NO. 35 | CCACAGA-BHQ-1 SEQ ID NO. 36 |

^{a}Human Genome Organisation gene name

### PAPPA and ZMPSTE24 Over-Expression in T47D Cells

PAPPA and ZMPSTE24 (control) are zinc-dependent metalloproteinases of the metzincin superfamily. Full-length human PAPPA cDNA (NM_002581.3) and ZMPSTE24 cDNA (NM_005857.2) were cloned into pCMV6-XL5 vectors (OriGene). Approximately 2×10⁶ T47D cells cultured in T75 flasks were transfected with 40 μg of PAPPA or ZMPSTE24 cDNA. Cells were collected 48 h and 72 h post-transfection. PAPPA and ZMPSTE24 expression levels were determined by qRT-PCR and western blot.

### RNAi-Rescue Experiments

Eight silent mutations were introduced into two separate sites of human PAPPA cDNA (NM_002581.3) within the regions targeted by two different PAPPA siRNAs (Ambion s10042 and 104028). The mutated cDNA was cloned into the pCMV6-XL5 vector (OriGene) and BT549 cells were transfected with 20 μg of the PAPPA rescue plasmid. Twenty-four hours after PAPPA-F+^{mut} overexpression, endogenous mRNA was knocked down using 100 nM PAPPA specific siRNA (Ambion s10042 or 104028). Cells were collected 48 h after knock down of endogenous mRNA.

### Invasion Assay

Invasion through extracellular matrix (ECMatrix) was measured in Boyden chamber assays (QCM Invasion Assay, Millipore) following the manufacturer's instructions. Briefly, BT549 cells were transfected with PAPPA ON-TARGETplus SMARTpool or control oligo in serum-free medium for 24 h. T47D cells were transfected with PAPPA or ZMPSTE24 expression constructs for 24 h prior starvation for 24 h in serum-free medium. BT549 and T47D cells were collected in RPMI medium containing 5% BSA, counted and 2.5×10⁵ cells were seeded in each invasion chamber. After incubation for 48 h (BT549) or 72 h (T47D) the invasion chamber inserts were washed with PBS, fixed in 4% paraformaldehyde for 5 min, stained with 0.1% crystal violet and cells from random areas on the filters were counted. Assays were performed in triplicate.

### β1-Integrin Cell Surface Expression

Live cells were immunostained in suspension (as described in Rizki, A. et al., J. Cancer research (2007) 67, 11106-11110), fixed in 2% PFA and FACS was performed as described in Rodriguez-Acebes et al, Am. J. Pathol, 2010; 177:2034-2045. The fluorescence peak was evaluated for its median value and corrected using samples, which had not been incubated with primary antibody (anti-CD29 HUTS-21, BD Pharmingen). To mask β1-integrin, 20 μg/ml of blocking antibody (anti-β1-integrin MAB1959, Chemicon; Vincourt, J. B. et al. (2010)Cancer research 70, 4739-4748) was added to the culture medium for the duration of the invasion assay.

### Statistical Analysis

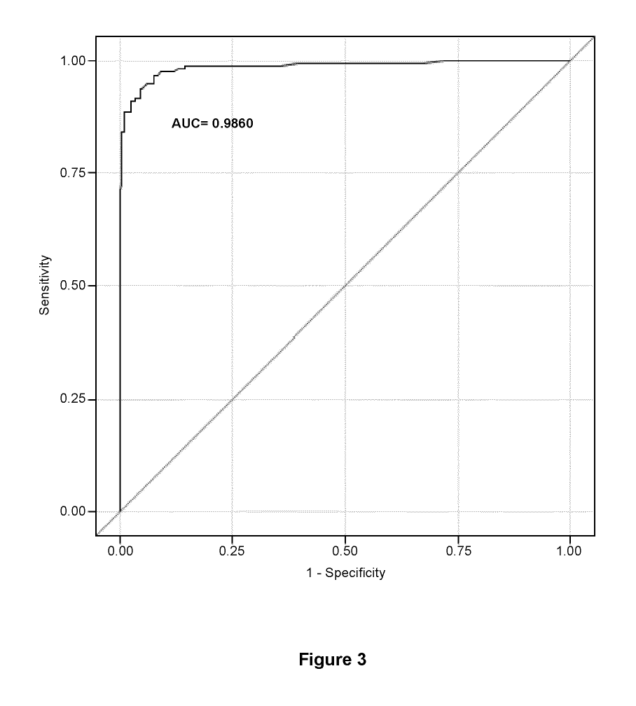

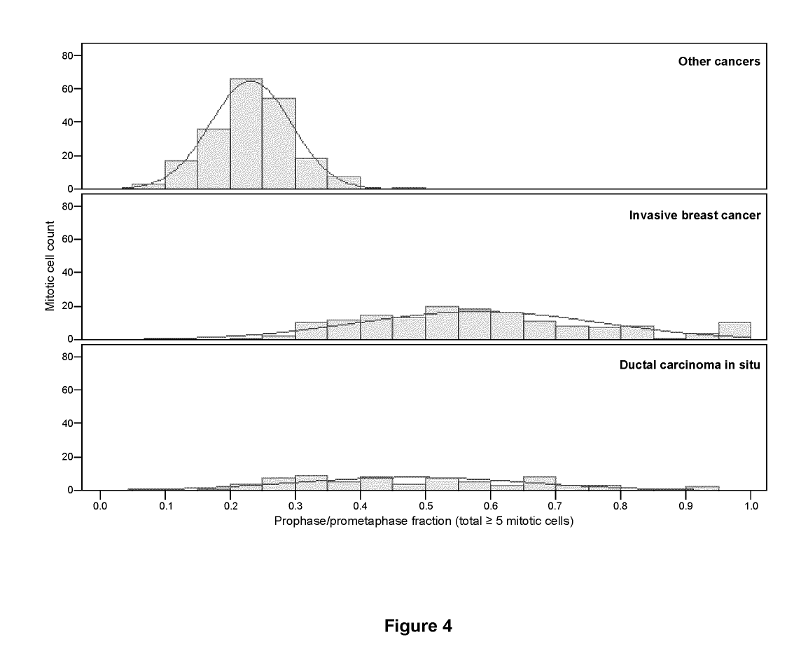

The proportion of mitotic cells in prophase/prometaphase was calculated for each specimen of premalignant and malignant tissue. Receiver Operating Characteristic (ROC) curves for differentiating breast cancer from other malignancies (pooled) using the proportion of cells in prophase/prometaphase were constructed for various minimum numbers of mitotic cells. It was clear that the ROC curve was not compromised by letting the minimum requirement be as low as five mitotic cells (FIG. 3). In the interest of using as many specimens as possible, the evaluability threshold for analysis of mitotic delay was set to at least five mitotic cells observed per specimen. A specimen was declared ‘delayed’ if at least one third of its mitotic cells were in prophase/prometaphase. This requirement was derived by balancing the sensitivity and specificity associated with distinguishing breast cancer from other malignancies: 94.9% of evaluable breast cancer specimens had at least one third of their mitotic cells in prophase/prometaphase, while 94.1% of evaluable other malignancies had less than one third of their mitotic cells in prophase/prometaphase (FIG. 4). The proportion of evaluable specimens with mitotic delay was compared between sources of specimen (breast cancer, DCIS, other malignancies) using Pearson's chi-squared test with Yates's continuity correction. The median proportion of mitotic cells in prophase/prometaphase was compared between sources of specimen using the Mann-Whitney test. All significance probabilities reported are two-sided.

The association of mitotic delay with tumour differentiation and nodal metastasis was assessed using a non-parametric Jonckheere-Terpstra test for trend, with morphological subtype and molecular subtype using a Krusal Wallis analysis of variance test, and with oestrogen/progesterone receptor status and aneuploidy using a Mann-Whitney test.

### Results

### Breast Cancer is Specifically Enriched in Early Mitotic Figures

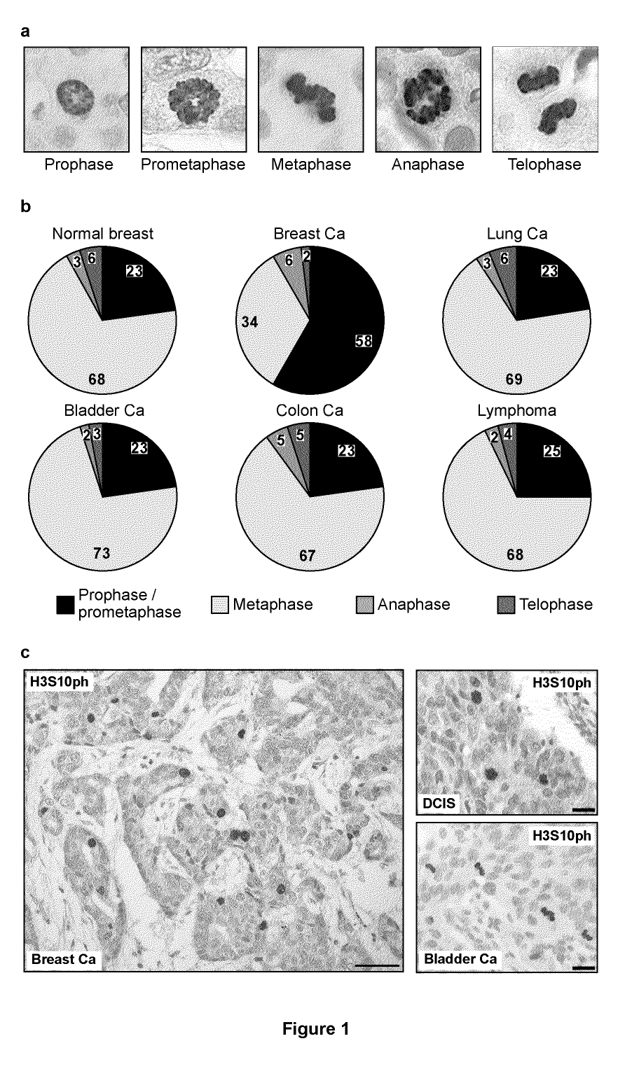

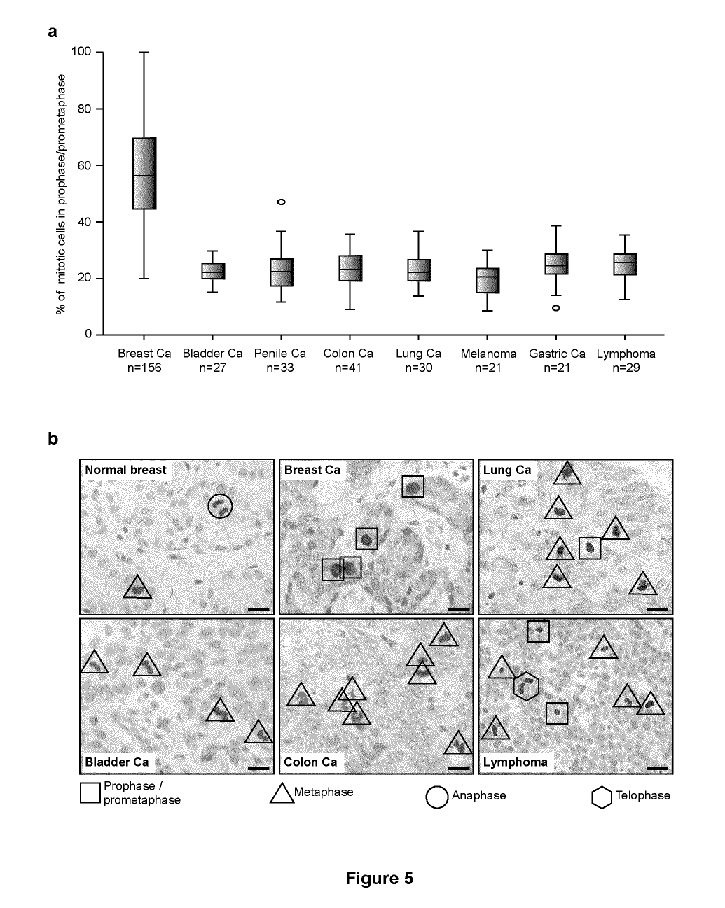

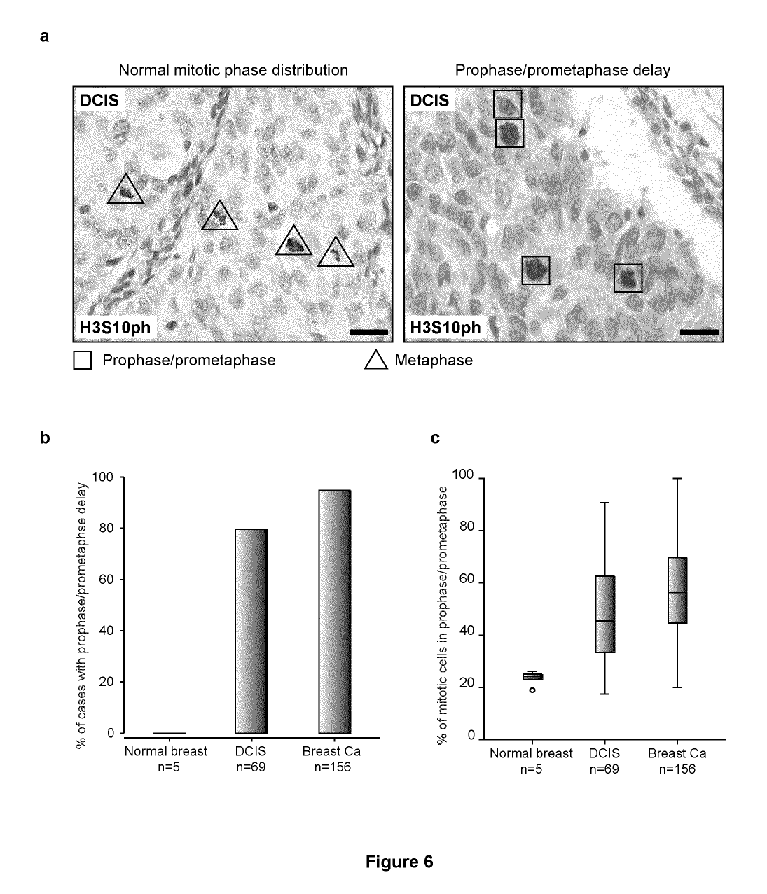

In evaluable tissue specimens from seven common human tumour types, including skin, lung, colon, gastric, bladder, penile and lymphatic cancer (n=202 patients), the majority of mitotic cells were in metaphase (FIG. 1a-b and FIG. 5a-b). In marked contrast the inventors found a strong prophase/prometaphase enrichment (defined as at least one third of mitotic cells in prophase/prometaphase) in 95% (148 out of 156 evaluable patients) of breast cancers compared with only 6% (12 out of 202) for the combined group of other malignancies (P<0.0001, Pearson's test with Yates's correction). The mean proportion of mitotic breast cancer cells in prophase/prometaphase was 58% (median 56%) compared with 23% (median 23%) in the other malignancies (P<0.0001, Mann-Whitney test) (FIG. 1b-c and FIG. 5a), indicating that an early mitotic delay or arrest is a hallmark of breast cancer. This was specific to diseased tissue, as normal proliferating breast tissue revealed an undisturbed mitotic phase distribution, again with 23% (median 24%) of mitotic cells residing in prophase/prometaphase (FIG. 1b and FIG. 5b). Importantly, the inventors found could detect a clear mitotic delay phenotype already in 80% (55 out of 69 evaluable patients) of non-invasive ductal carcinoma in situ (DCIS) lesions (P<0.0001 compared with the group of other malignancies), in which the mean proportion of mitotic cells in prophase/prometaphase was 48% (median 45%) (P<0.0001 compared with the group of other malignancies) (FIG. 1c and FIG. 6). Thus the tumour screen for specific mitotic phenotypes, first seen by gene silencing in a cell culture model (Neumann, B. et al. Nature (2010) 464, 721-727), identified an unexpectedly high frequency of early mitotic figures (prophase/prometaphase) in nearly all tested breast cancers, revealing a formerly unrecognized delay in mitotic progression in this tumour type.

MitoCheck Hits with a Breast Cancer-Like Mitotic Phenotype

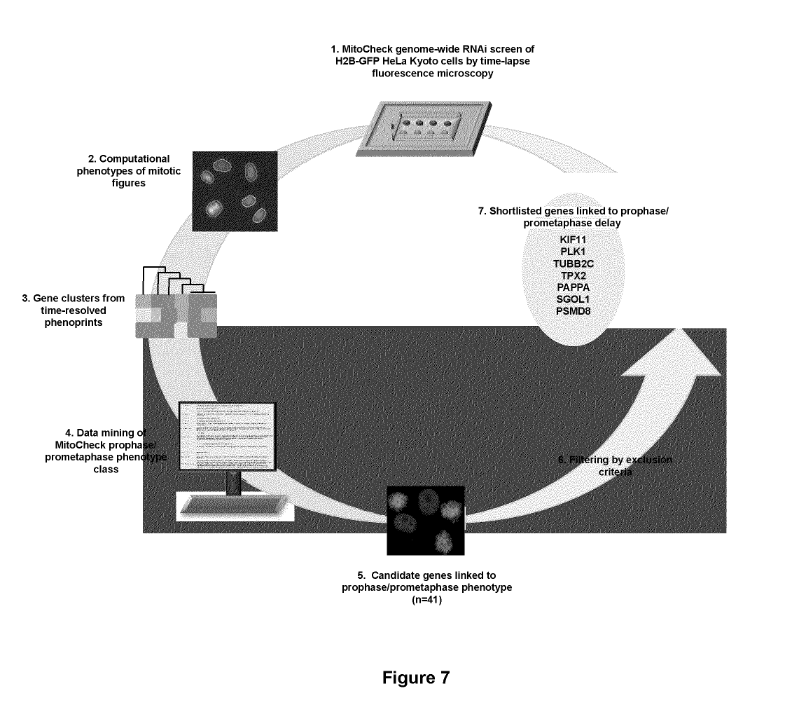

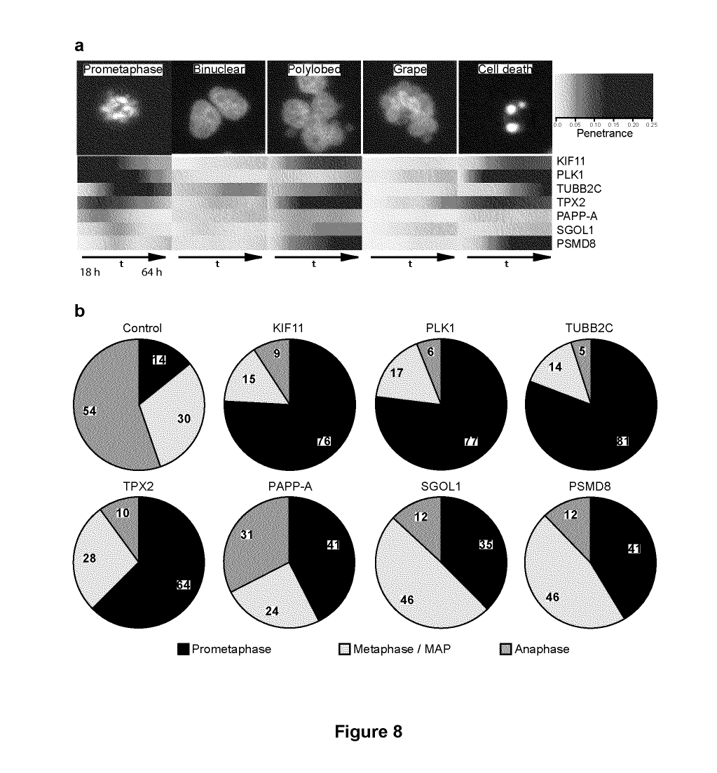

Early mitotic delay was a very specific and relatively rare mitotic phenotype in the MitoCheck screen (Neumann, B. et al.). In order to identify candidate genes whose down-regulation in cultured human cells results in a similar early mitotic phenotype to the one the inventors had observed in breast cancer, they searched the MitoCheck database (accessible at www.mitocheck.org), focusing specifically on the prophase/prometaphase class, which was morphologically most similar to the phenotypes that had been observed by the inventors in breast cancer tissues (FIG. 7). In the genome wide data set KIF11, PLK1, TUBB2C, TPX2, PAPPA, SGOL1 and PSMD8 displayed a significant increase in the prophase/prometaphase class (FIG. 8), indicating a delay or arrest in prophase/prometaphase, as detected in breast cancer. The quantitative scoring of the time-resolved phenotypic answers to the knock down of these individual genes showed for all seven genes as a primary phenotype prometaphase arrest, followed by secondary phenotypes (e.g. cell death or polylobed nuclear shape) (FIG. 8). RNAi experiments targeting KIF11, PLK1, TUBB2C and TPX2 showed a high percentage of cells in prometaphase already 12 to 25 hours after transfection, while PAPPA, SGOL1 and PSMD8 knock downs caused lower prometaphase phenotype penetrance but had an overall similar phenotypic profile (FIG. 8). Cell death as a consequence of the mitotic phenotype was significant for all genes, except for PAPPA and SGOL1 (FIG. 8), making them the strongest candidates for tumour suppressor genes whose mitotic aberrations are not expected to be cleared by cell death.

### PAPPA Loss is Linked to Mitotic Delay in Breast Cancer

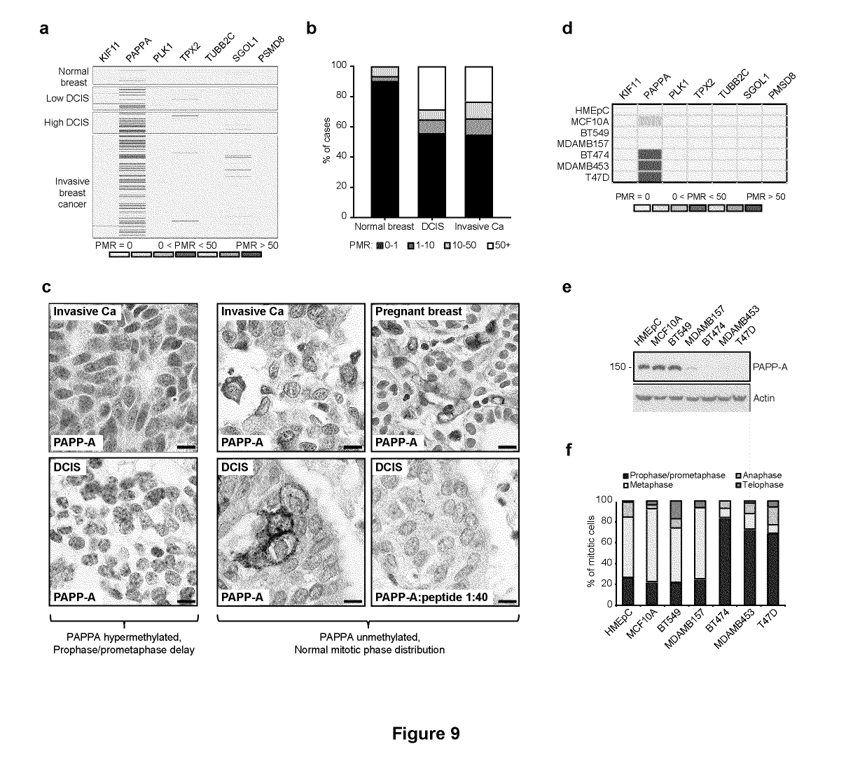

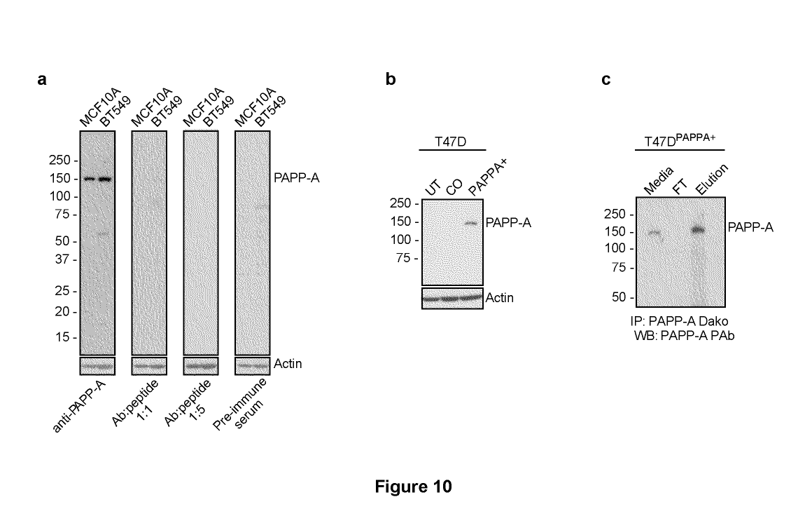

Since promoter methylation represents a common mechanism for loss-of-function of tumour suppressors during cancer development, the inventors hypothesised that epigenetic silencing of any of the seven MitoCheck candidate genes could be linked to the mitotic delay phenotype which the inventors found in breast cancer. Indeed, MethyLight assays (Widschwendter, M. et al. Cancer research (2004) 64, 3807-3813) showed that of the seven candidate genes only PAPPA is strongly hypermethylated in the 5′ regulatory region of the gene in invasive breast cancers and in non-invasive DCIS lesions (FIG. 9a). Forty-six % (80 out of 173 patients assessed for methylation) of breast cancers and 45% (34 out of 75 assessed patients) of DCIS lesions showed PAPPA hypermethylation (PMR>1; percentage methylated reference gene). In contrast, PAPPA was unmethylated in the majority of normal breast tissue samples (27 out of 30 assessed patients) (FIG. 9b; note that nine cases of breast cancer, six cases of DCIS and four cases of normal breast were not available for MethyLight analysis due to poor preservation of DNA). This made PAPPA the strongest candidate to explain the strong prophase/prometaphase delay found in breast cancer. To test if PAPPA promoter methylation indeed caused gene silencing, the inventors used a commercially available anti-PAPPA antibody (DAKO) for immuno-labelling of tissue sections and an affinity-purified rabbit anti-PAPPA PAb (FIG. 10) for western blotting. Immuno-expression analysis showed that indeed 96% (81 out of 84 patients) of non-invasive and invasive breast cancers with methylated PAPPA promoter and exhibiting the mitotic delay phenotype were not expressing PAPPA protein (FIG. 9c). By contrast, the majority of non-delayed/PAPPA unmethylated breast cancers (73%, 7 out of 11 patients), as well as normal proliferating pregnant breast tissue (n=5 patients) showed strong PAPPA immuno-staining predominantly at the cell membrane, consistent with a secreted protein (FIG. 9c).

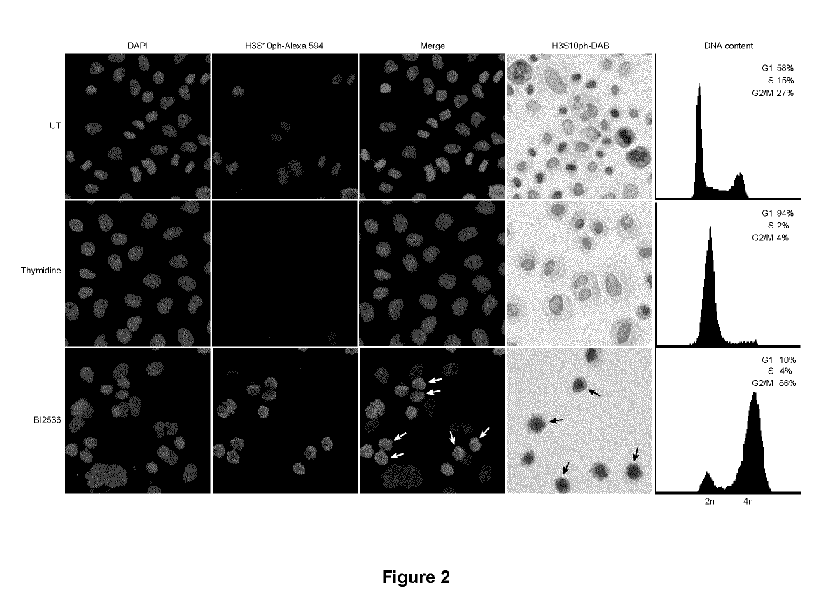

Validating the hypothesis that loss of PAPPA expression causes the mitotic delay in breast cancer requires an experimentally accessible system. The inventors therefore investigated whether the linkage between PAPPA gene silencing and the mitotic delay phenotype has been maintained in cultured breast cells. Consistent with the inventors' in vivo findings, cells with unmethylated PAPPA promoter and detectable PAPPA protein, including primary human mammary epithelial cells (HMEpC), immortalized MCF10A cells and the BT549 and MDAMB157 breast cancer cell lines, showed a normal mitotic phase distribution (FIG. 9d-f), as determined by morphological analysis of cytospin preparations immuno-labelled with the same phosphohistone H3 (H3S10ph) antibody used for the tissue screen (FIG. 2). By contrast, cell lines with heavily methylated PAPPA promoter (PMR>50) and strongly reduced PAPPA protein, including the BT474, MDAMB453 and T47D breast cancer cell lines, all exhibited the mitotic delay phenotype with ˜80% of mitotic cells residing in prophase/prometaphase (FIG. 9d-f).

### PAPPA is Required for Early Mitotic Progression in Breast Cancer Cells

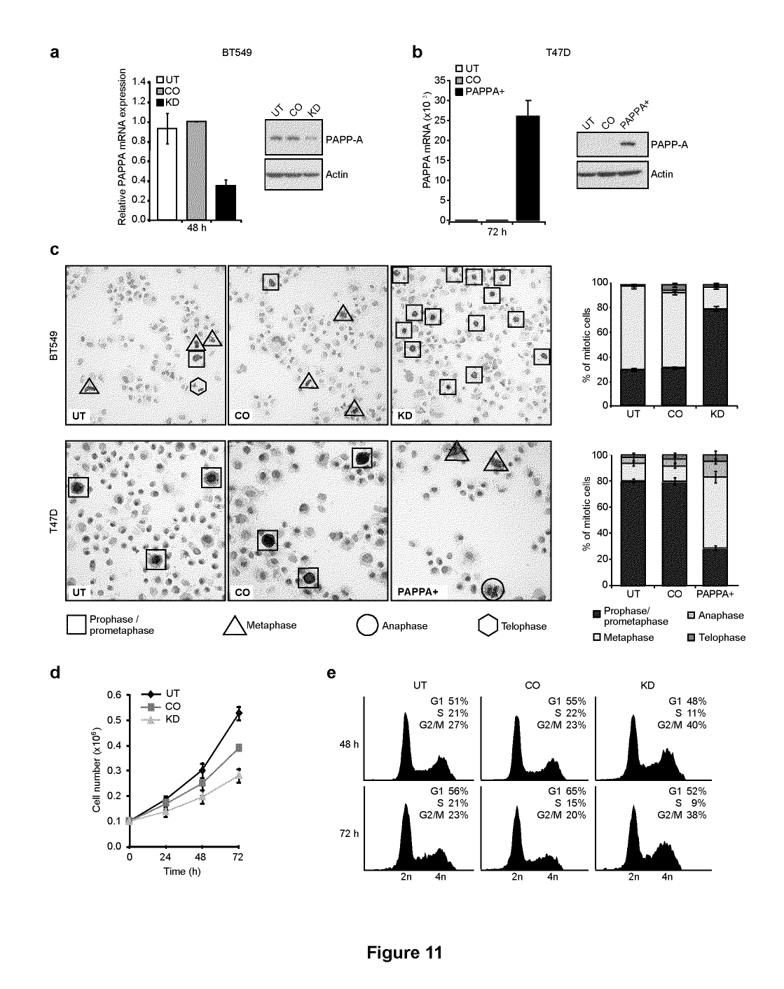

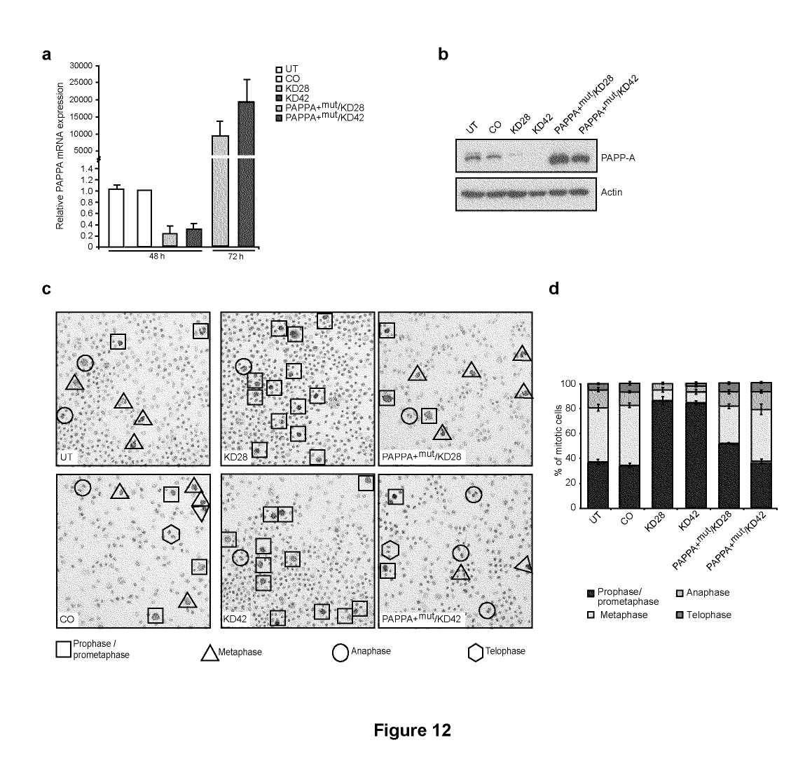

Having breast cancer cell lines in hand that recapitulate the link between loss of PAPPA expression and mitotic delay observed in patient tissue, the inventors first investigated whether PAPPA knock down by RNAi induces prophase/prometaphase delay in BT549 cells, in which the gene is not silenced through promoter methylation (FIG. 9e-f). Relative to control-siRNA, transfection of BT549 cells with a pool of four RNA duplexes targeting PAPPA mRNA reduced transcript levels by ˜70% and PAPPA protein levels by ˜60% (FIG. 11a). Analysis of BT549 cytospin preparations immuno-labelled for phosphohistone H3 (H3S10ph) revealed that, indeed, PAPPA knock down induced a strong prophase/prometaphase delay phenotype in this cell line (FIG. 11c), very similar to the phenotype observed in HeLa cells in the MitoCheck screen (Neumann, B. et al. Nature (2010) 464, 721-727). Consistent with the increased proportion of mitotic BT549 cells in prophase/prometaphase, PAPPA knock down caused a marked increase in the cell population doubling time (FIG. 11d) with a concomitant ˜2-fold increase in the proportion of cells with G2/M DNA content (FIG. Ile) and a 3-fold increase in the mitotic index. The inventors verified the prophase/prometaphase delay phenotype caused by PAPPA depletion by transfecting BT549 cells with single siRNA duplexes (siRNA-28 and siRNA-42) targeting different regions of the transcript (FIG. 12). To confirm that the RNAi phenotype was specifically due to PAPPA depletion, the inventors overexpressed a PAPPA cDNA variant resistant to siRNA-28 and siRNA-42. Expression of this construct restored PAPPA protein expression and rescued the mitotic delay phenotype caused by either of the two single siRNA duplexes (FIG. 12). If PAPPA loss causes the mitotic delay phenotype in breast cancer cells with hypermethylated PAPPA promoter such as T47D cells (FIG. 9d-e), the inventors reasoned that in this experimental system PAPPA overexpression should rescue the phenotype. Indeed, transfection of T47D cells with PAPPA cDNA (PAPPA+) restored PAPPA mRNA and protein levels (FIG. 11b) and fully reversed the mitotic delay phenotype, resulting in a mitotic phase distribution very similar to BT549 cells with normal PAPPA expression (FIG. 11c). Taken together, these results show that PAPPA is required for progression through early mitosis and that PAPPA down-regulation through epigenetic silencing (T47D cells) or experimentally by RNAi (BT549 cells) causes a strong prophase/prometaphase delay phenotype.

### PAPPA Loss Increases Invasiveness of Breast Cancer Cells

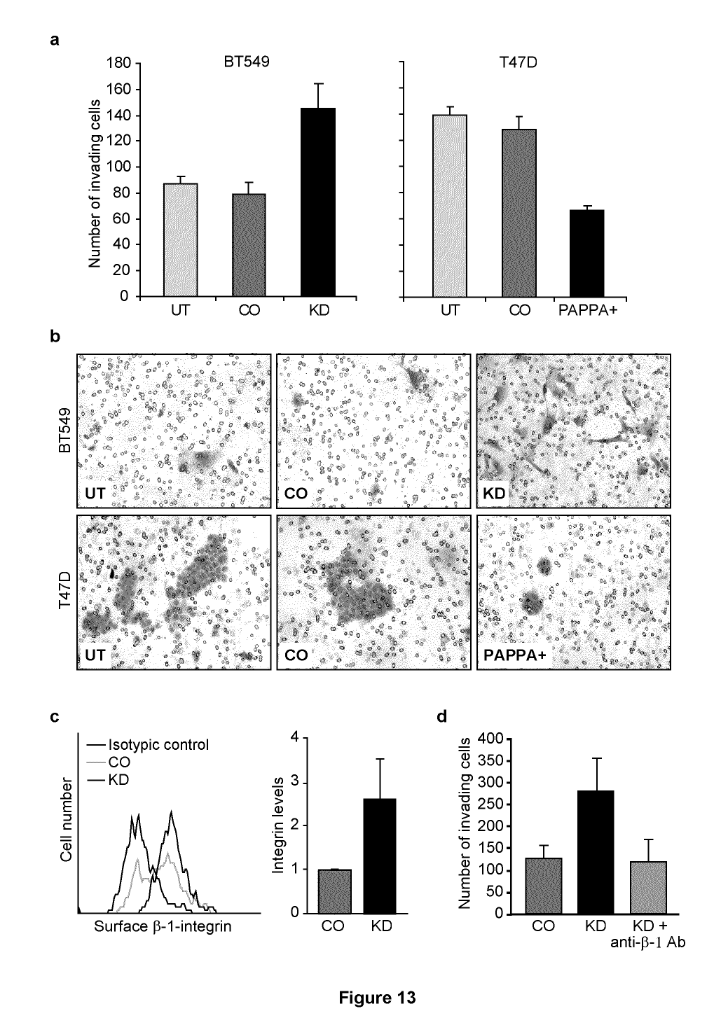

Next the inventors asked what biological advantage is conferred to the neoplastic breast cell through perturbation of early mitotic progression. To address this question they started by looking for any linkages between the mitotic delay phenotype and clinico-pathological features determined for each breast cancer specimen during routine clinical investigation (n=156 evaluable patients). This analysis revealed no linkage between mitotic delay and tumour differentiation (grade), morphological subtype (invasive ductal, lobular, mixed, mucinous, and micropapillary), molecular subtype (luminal, Her-2 or triple negative/basal-like), oestrogen/progesterone receptor status, nodal metastasis or aneuploidy. Notably, though, the presence of the mitotic delay phenotype in nearly all invasive breast cancer specimens studied (95%, 148 out of 156 evaluable patients) but in only a proportion of non-invasive DCIS lesions (80%, 55 out of 69) raises the possibility that this mitotic defect might be linked to the acquisition of invasiveness. To test this particular hypothesis, the inventors induced the mitotic delay phenotype in BT549 cells by PAPPA knock down (alternatively normal mitotic progression was restored in T47D cells by exogenous PAPPA expression) and measured the invasiveness of the manipulated cells in Matrigel-coated Boyden chamber assays. In parallel, the inventors used flow cytometry to determine the cell surface levels of β1-integrin, a well-characterised invasion marker in breast cancer. Silencing of PAPPA was associated with a marked (2-fold) increase in the number of invading cells (FIG. 13a-b). The increased invasiveness of PAPPA depleted BT549 cells was also mirrored by an increase in β1-integrin cell surface levels (FIG. 13c). Notably, the increase in invasiveness associated with PAPPA knock down was fully reversed following treatment of BT549 cells with a β1-integrin blocking antibody prior to transfer of the cells to the Boyden chamber (FIG. 13d). Conversely, re-establishment of normal mitotic phase distribution by exogenous PAPPA expression strongly reduced the invasiveness of T47D cells (FIG. 13a-b). These results demonstrate that loss of PAPPA function delays progression through early mitosis and increases the capacity of breast cancer cells to become more invasive.

### Example 2

### Methylation as Molecular Signature for Breast Cancer in Liquid Biopsies

### Materials

Normal control plasma samples were obtained from a commercial source (Tissue Solutions, Glasgow, UK). Blood samples from metastatic breast cancer patients were sourced from Royal National Orthopaedic Hospital, London, UK following institutional ethical guidelines. Samples used in this study include healthy female control samples (n=3), blood samples from breast cancer patients (n=12), and one plasma sample (n=1) from a breast cancer patient. Sample characteristics are listed in Table 3. All use of, or transfer of, blood or blood products was performed in accordance with Human Tissue Authority (HTA) Guidelines.

TABLE 3

| | Sample | | Site of | Multiple/single | |
|---|---|---|---|---|---|
| Sample | ID | Type | metastasis | metastatic sites | Description |
| Control | N1 | Normal female | N/A | N/A | Human plasma |
| Control | N2 | Normal female | N/A | N/A | Human plasma |
| Control | N3 | Normal female | N/A | N/A | Human plasma |
| Breast cancer | BC1a | Metastatic | Right femur | Multiple | Human plasma |
| Breast cancer | BC1 | Metastatic | Right femur | Multiple | Human whole |
| | | | | | blood |
| Breast cancer | BC2 | Metastatic | Left ilium | Single | Human whole |
| | | | | | blood |
| Breast cancer | BC3 | Metastatic | Humerus | Single | Human whole |
| | | | | | blood |
| Breast cancer | BC4 | Metastatic | Left femur | Multiple | Human whole |
| | | | | | blood |
| Breast cancer | BC5 | Metastatic | Left femur | Multiple | Human whole |
| | | | | | blood |
| Breast cancer | BC6 | Metastatic | Left femur | Multiple | Human whole |
| | | | | | blood |
| Breast cancer | BC7 | Metastatic | Right pelvis | Multiple | Human whole |
| | | | | | blood |
| Breast cancer | BC8 | Metastatic | Pelvis, humerus | Multiple | Human whole |
| | | | | | blood |
| Breast cancer | BC9 | Metastatic | Left femur | Multiple | Human whole |
| | | | | | blood |
| Breast cancer | BC10 | Metastatic | Right femur | Possible multiple | Human whole |
| | | | | (left femur) | blood |
| Breast cancer | BC11 | Metastatic | Humerus | Multiple | Human whole |
| | | | | | blood |
| Breast cancer | BC12 | Metastatic | Vertebra | Multiple | Human whole |
| | | | | | blood |

### Methods

Plasma Preparation from Whole Blood Samples

Whole blood samples (containing EDTA as anti-coagulant) were thawed and plasma prepared by centrifugation at 1,000×g for 10 min at room temperature. The supernatant was aliquoted and stored at −80° C.

### DNA Isolation Using the Norgen Plasma Circulating DNA Kit

Prior to DNA isolation, plasma samples were centrifuged at 16,000×g for 5 min to remove remaining cells and debris. Circulating genomic DNA was isolated from all plasma samples using the Norgen Plasma/Serum Circulating DNA Isolation Mini Kit (Geneflow, Cat no. P4-0124, Lot no. 583601) according to the manufacturer's instructions. Circulating DNA was isolated from plasma in two preparation runs of 400 μl per sample. The final elution step was performed with 50 μl elution buffer.

### Bisulfite Modification

DNA isolated from plasma was bisulfite modified using the EZ-DNA Methylation Gold Kit (Zymo Research, Cat no. D5006, Lot no. ZRC175490) according to the manufacturer's instructions. CpG methylated HeLa genomic DNA (New England Biolabs, Cat no. 4007S, Lot no. 51301) was treated in parallel as a reference for total and PAPPA promoter methylated DNA. 100 μL CT conversion reagent was added to 50 μL of DNA isolation eluate. Samples were incubated at 98° C. for 10 min, then at 64° C. for 2.5 h on a MJ Mini 48-Well Personal Thermal Cycler (Bio-Rad, Cat no. PTC-1148). Samples were processed, purified on a spin column and eluted. Bisulfite-modified DNA was analysed by MethyLight assay or stored at −80° C. until required.

### MethyLight Assay PCR

Two sets of primers and probes were designed for bisulfite-modified DNA: a methylated set for PAPPA and a set for collagen 2A1 (COL2A1) to normalise for input DNA. The primer stes are shown in Table 4. q-PCR reactions were carried out using the TaqMan® Universal PCR Master Mix [No AmpErase® UNG (Life technologies, Cat no. 4324018, Lot no. S12230)] with 2 μl of the eluted DNA, or H₂O as negative control, 0.3 μM probe and 0.9 μM of both forward and reverse primer (shown below). The reactions were carried out in triplicate on a StepOne Plus Real Time PCR system (Life Technologies, Model No. 272006346). The cycling conditions were: 95° C. (10 min), followed by 60 cycles of 95° C. (15 s), 60° C. (1 min). The results of the PCR reaction were analysed (DDCT calculations) using StepOne software V2.3.

TABLE 4

| | Forward primer | Reverse primer | |
|---|---|---|---|
| Gene | sequence 5′-3′ | sequence 5′-3′ | Probe sequence 5′-3′ |
| PAPPA | GCGTGTTTGTGCGAGAG | CGCCTTCCGAATATACCC | 6-FAM- |
| | TTGT (SEQ ID NO. 7) | ATT (SEQ ID NO. 8) | TCGCCCGAATATCTCTACGCCGCT- |
| | | | BHQ-1 (SEQ ID NO. 9) |
| COL2A1 | TCTAACAATTATAAACTC | GGGAAGATGGGATAGAA | 6-FAM- |
| | CAACCACCAA | GGGAATAT | CCTTCATTCTAACCCAATACCTATCCC |
| | (SEQ ID NO. 31) | (SEQ ID NO. 32) | ACCTCTAAA-BHQ-1 (SEQ ID NO. 33) |

### Results

Methylation of the PAPPA Promoter can be Specifically Detected in Circulating DNA from Blood of Breast Cancer Patients

Previous studies carried out by the inventors indicate that epigenetic silencing of PAPPA by promoter hypermethylation is a feature of breast cancer. In keeping with this finding, PAPPA promoter hypermethylation was detected in invasive breast cancer tumour tissues compared to normal breast tissue specimens. Based on these findings the inventors hypothesized that circulating tumour DNA carrying PAPPA promoter hypermethylation as a genetic alteration can be detected in blood samples from breast cancer patients.

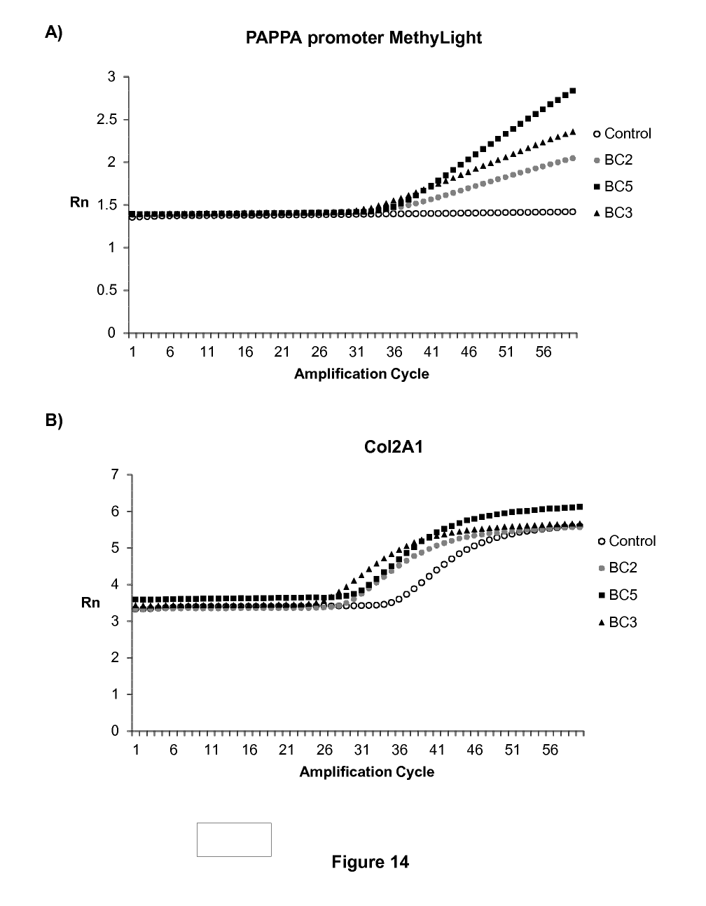

FIG. 14A shows a representative MethyLight assay amplification using DNA purified from plasma of three breast cancer samples and one healthy control. PAPPA promoter hypermethylation was detected in blood of breast cancer patients but not in control samples. FIG. 14B demonstrates, using the amplification of the endogenous control gene COL2A1, that DNA could be purified from all samples.

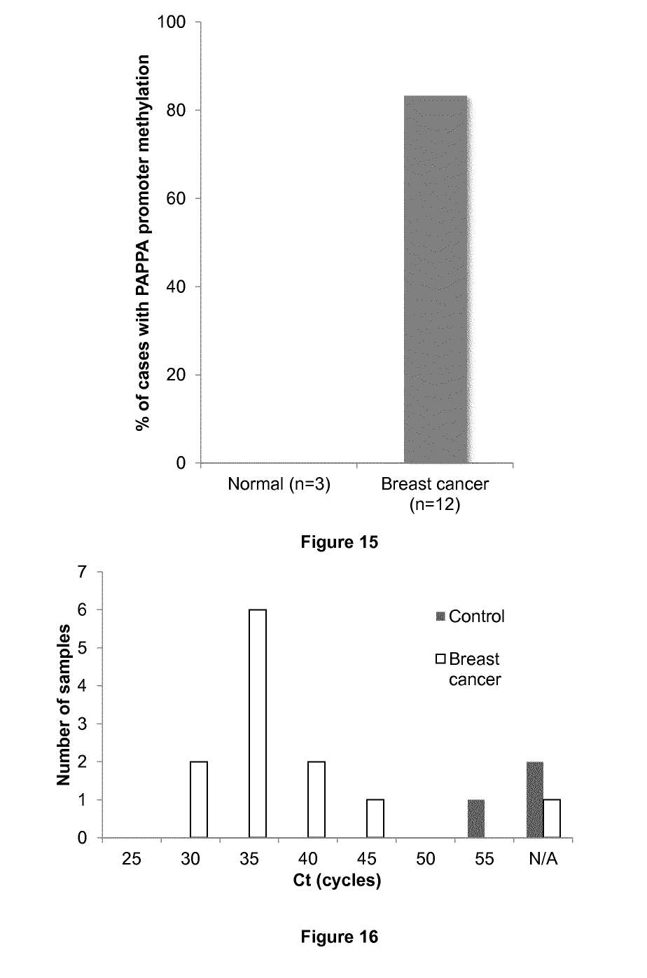

FIG. 15 shows the percentage of samples from breast cancer patients and healthy female controls, in which PAPPA promoter methylation was detectable above the amplification threshold by MethyLight assay. As the samples used in the study were frozen blood samples, it can be postulated that DNA from lysed cellular components of blood was released which may decrease the sensitivity of the MethyLight assay. Hence, the data obtained by MethyLight assay were analysed by arbitrarily setting a mean Ct value threshold of 45 cycles. Any amplification signal above the threshold was discounted for the analysis. Using this threshold, PAPPA promoter methylation was detected in 83% of the samples (10 out of 12) in contrast to none of the control samples. A Fisher's exact test of breast cancer samples against the healthy controls yielded a p value of 0.022.

FIG. 16 shows the distribution of the Ct values for the individual breast cancer samples versus control samples.

### DISCUSSION

Promoter methylation represents a common mechanism for loss of tumour suppressor genes during cancer development. The inventors previously established that epigenetic silencing of PAPPA is causally connected with breast cancer tumorigenesis. Previous studies showed that the PAPPA gene is strongly hypermethylated in the 5′ regulatory region of the gene in invasive breast cancer and correlating with a loss of PAPPA protein expression. Using DNA extracted from formalin fixed paraffin embedded tissue blocks, it was shown that 46% (80 out of 173 patients) of invasive breast cancers, showed PAPPA hypermethylation. In contrast, PAPPA was unmethylated in 90% (27 out of 30 cases) of normal breast samples. In this study, the inventors claim that an enhanced level of DNA containing methylation of the PAPPA promoter can be detected in the blood of breast cancer patients using a MethyLight assay.

Compared to tissue biopsies, blood samples (liquid biopsies) can be easily obtained, and are therefore a suitable sample type for cancer screening. Circulating DNA is found in the plasma of healthy individuals in the range of 1-100 ng/ml and can be increased in cancer patients due to lysis of circulating tumour cell, or enhanced cell lysis in tumour regions. Circulating tumour DNA has been tested for tumour specific epigenetic changes such as DNA methylation (Board et al, Biomarker Insights (2), 2007). Notably, DNA methylation in the plasma has been described to correlate to the same epigenetic alterations found in the tumour biopsies (Skvortsova et al, Br J Cancer 94, 2006).

The method described here can be used as an aid in staging patients diagnosed with breast cancer. The method can be used to assess invasion of breast cancer cells into neighbouring tissue or spread to distant sites by monitoring the level of loss-of-function related genetic alterations in PAPPA. The method described here can also be used for monitoring the response to treatment in patients undergoing breast cancer therapy, comprising detecting the presence of a loss-of-function related genetic alteration in the PAPPA gene or its regulatory or promoter sequences in a sample obtained from a patient wherein if the genetic alteration is present but its quantitative level decreases during treatment, this is indicative of response to therapy, whereas no change or an upward change in its quantitative level is indicative of non-response to therapy. Moreover, this method can also determine if a patient previously subjected to breast cancer therapy has suffered a relapse. The method described in this patent can also be used for blood based screening of asymptomatic subjects wherein the presence of loss-of-function related genetic alteration suggests an increased risk of breast cancer.

Taken together, the results presented here show that PAPPA promoter methylation can be used as a molecular signature for breast cancer screening in asymptomatic subjects, staging of invasive breast cancer patients, monitoring efficacy to treatment regimens and relapse monitoring in invasive breast cancer patients. The invention described in this patent may allow more accurate staging of breast cancer, and effective monitoring of treatment response and early relapse before it is detected by standard tests in the clinic.

1. A method for monitoring the response to treatment in a patient undergoing breast cancer therapy, comprising detecting the presence of a loss-of-function-related genetic alteration in the PAPPA gene or its regulatory or promoter sequences in a sample obtained from the patient,
wherein if the genetic alteration is present but its quantitative level decreases during treatment, this is indicative of response to therapy, whereas if there is no change or upward change in its quantitative level during therapy, this is indicative of non-response to therapy.

2. A method according to claim 1, wherein the loss-of-function-related genetic alteration in the PAPPA gene or its regulatory or promoter sequences is one or more of methylation, deletion, a point mutation, loss-of-heterozygosity, translocation, insertion or chromosomal breakage.

3. A method for monitoring the response to treatment in a patient undergoing breast cancer therapy, comprising detecting the presence and/or level of PAPPA in a sample obtained from the patient,
wherein the absence of PAPPA, or PAPPA activity, or the presence of PAPPA, or PAPPA activity at a reduced level compared to a control is indicative of non-response to therapy.

4. A method according to any of claims 1 to 3, wherein the sample is a blood sample.

5. A method for determining whether a patient, previously treated with breast cancer therapy, has suffered a relapse, comprising detecting the presence of a loss-of-function-related genetic alteration in the PAPPA gene or its regulatory or promoter sequences in a biological sample obtained from the patient,
wherein the presence of a genetic alteration is indicative of a relapse.

6. A method according to claim 5, wherein the loss-of-function-related genetic alteration in the PAPPA gene or its regulatory or promoter sequences is one or more of methylation, deletion, a point mutation, loss-of-heterozygosity, translocation, insertion or chromosomal breakage.

7. A method for determining whether a patient previously treated with breast cancer therapy, has suffered a relapse, comprising detecting the presence and/or level of PAPPA in a sample obtained from the patient,
wherein the absence of PAPPA, or PAPPA activity, or the presence of PAPPA, or PAPPA activity at a reduced level compared to a control, is indicative of a relapse.

8. A method according to any of claims 5 to 7, wherein the sample is blood, tissue biopsy or nipple aspirates.

9. A method for determining whether a patient previously treated with breast cancer therapy has suffered a relapse, comprising
identifying the proportion of mitotic cells in a tissue sample obtained from the patient that are in prophase or pro-metaphase and comparing to a pre-determined cut-off value, wherein if the proportion of cells in prophase or pro-metaphase is the same or greater than the cut-off value there is a risk that the patient has suffered a relapse.

10. A method for determining whether a primary breast cancer in a patient has spread away from the primary tumour to other parts of the body or infiltrated tissue adjacent to the primary tumour, comprising detecting the presence of a loss-of-function-related genetic alteration in the PAPPA gene or its regulatory or promoter sequences in a sample obtained from the patient,
wherein the presence of a genetic alteration is indicative of spread or infiltration, and wherein the sample is a blood sample or tissue sample adjacent to or distant from the site of the primary tumour.

11. A method according to claim 10, wherein the loss-of-function-related genetic alteration in the PAPPA gene or its regulatory or promoter sequences is one or more of methylation, deletion, a point mutation, loss-of-heterozygosity, translocation, insertion or chromosomal breakage.

12. A method for determining whether a primary breast cancer in a patient has spread away from the primary tumour to other parts of the body or infiltrated tissue adjacent to the primary tumour, comprising identifying the proportion of mitotic cells in a tissue sample obtained from the patient that are in prophase or pro-metaphase and comparing to a pre-determined cut-off value,
wherein if the proportion of cells in a prophase or pro-metaphase is the same or greater than the cut-off value there is a risk that the cancer has spread or infiltrated, wherein the tissue sample is from tissue adjacent to or distant from the site of the primary tumour.

13. A method for determining whether a primary breast cancer in a patient has spread away from the primary tumour to other parts of the body or infiltrated tissue adjacent to the primary tumour, comprising detecting the presence and/or level of PAPPA in a sample obtained from the patient,
wherein the absence of PAPPA, or PAPPA activity or the presence of PAPPA, or PAPPA activity, at a reduced level compared to a control, is indicative of spread or infiltration, wherein the sample is a blood sample or tissue sample adjacent to or distant from the site of the primary tumour.

14. A method for screening for breast cancer in a subject, comprising detecting the presence of a loss-of-function-related genetic alteration in the PAPPA gene or its regulatory or promoter sequences in a blood sample obtained from the subject, wherein the presence of a genetic alteration is indicative of breast cancer.

15. A method according to claim 14, wherein the loss-of-function-related genetic alteration in the PAPPA gene or its regulatory or promoter sequences is one or more of methylation, deletion, a point mutation, loss-of-heterozygosity, translocation, insertion or chromosomal breakage.

16. A method for screening for breast cancer in a subject, comprising detecting the presence and/or level of PAPPA in a blood sample obtained from the subject, wherein the absence of PAPPA or PAPPA activity in the sample, or the presence of PAPPA, or PAPPA activity, at a reduced level compared to a control, is indicative of breast cancer.

17. A method according to any of claims 14 to 16, wherein the patient is presenting for routine screening and is asymptomatic for breast cancer.

18. A method for aiding primary diagnosis of breast cancer in a patient, comprising detecting the presence of a loss-of-function-related genetic alteration in the PAPPA gene or its regulatory or promoter sequences in a sample obtained from the patient,
wherein the presence of a genetic alteration is indicative of breast cancer, and wherein the sample is selected from blood and nipple aspirates.

19. A method according to claim 18, wherein the loss-of-function-related genetic alteration in the PAPPA gene or its regulatory or promoter sequences is one or more of methylation, deletion, a point mutation, loss-of-heterozygosity, translocation, insertion or chromosomal breakage.

20. A method for aiding primary diagnosis of breast cancer in a patient, comprising detecting the presence and/or level of PAPPA, or PAPPA activity, in a sample obtained from the patient,
wherein if PAPPA, or PAPPA activity, is not present, or is present at a reduced level compared to a control, the result is indicative of breast cancer, and wherein the sample is selected from blood and nipple aspirates.

21. A method according to any of claims 18 to 20, wherein the patient is presenting with symptoms of breast cancer.

22. A method according to any of claims 3, 7, 13, 16 and 20, wherein the presence of PAPPA is identified using a PAPPA-specific antibody or a probe for the PAPPA gene, mRNA or a specific PAPPA mutation.

23. A method according to any of claims 9 and 12, wherein the cut-off value is at least 30% of the mitotic cells in the sample.

24. A method according to any of claims 9, 12 and 23, wherein at least five of the cells in the sample are in mitosis.

25. A method according to any of claims 9, 12, 23 and 24, wherein the proportion of cells that are in prophase or pro-metaphase is determined using immuno-detection.

26. A method according to claim 25, wherein immuno-detection is carried out using an H3S10ph antibody.


SEQUENCE LISTING (44 sequences)

SEQ ID NO: 1 - DNA, 21 bp, Artificial
  Synthetic primer sequence
cgagcgttgtatgttgggatt

SEQ ID NO: 2 - DNA, 22 bp, artificial
  Synthetic primer sequence
cgcaacgaacgataacatctca

SEQ ID NO: 3 - DNA, 20 bp, Artificial
  Synthetic probe sequence
aactacgcaaacatccgccg

SEQ ID NO: 4 - DNA, 24 bp, Artificial
  Synthetic primer sequence
gcgtcgaggtttttaaagttggta

SEQ ID NO: 5 - DNA, 21 bp, Artificial
  Synthetic primer sequence
cccaactccaaaaccgcatat

SEQ ID NO: 6 - DNA, 19 bp, Artificial
  Synthetic probe sequence
ccctacaccgccacccgaa

SEQ ID NO: 7 - DNA, 21 bp, Artificial
  Synthetic primer sequence
gcgtgtttgtgcgagagttgt

SEQ ID NO: 8 - DNA, 21 bp, Artificial
  Synthetic primer sequence
cgccttccgaatatacccatt

SEQ ID NO: 9 - DNA, 24 bp, Artificial
  Synthetic probe sequence
tcgcccgaatatctctacgccgct

SEQ ID NO: 10 - DNA, 20 bp, Artificial
  Synthetic primer sequence
gttcgggcgttcgtgttaat

SEQ ID NO: 11 - DNA, 17 bp, Artificial
  Synthetic primer sequence
gccgcgcaacaccataa

SEQ ID NO: 12 - DNA, 28 bp, Artificial
  Synthetic probe sequence
ccctacgcaacaacaacctttaaacccg

SEQ ID NO: 13 - DNA, 23 bp, Artificial
  Synthetic primer sequence
ggttttatcggcgaaagagattt

SEQ ID NO: 14 - DNA, 17 bp, Artificial
  Synthetic primer sequence
cgaccccgcacataacg

SEQ ID NO: 15 - DNA, 26 bp, Artificial
  Synthetic probe sequence
ccgactacgtaaatccactaaaacct

SEQ ID NO: 16 - DNA, 23 bp, Artificial
  Synthetic primer sequence
cgcgtttcgttgttagatttgag

SEQ ID NO: 17 - DNA, 20 bp, Artificial
  Synthetic primer sequence
aaaaacccgcgccctaacta

SEQ ID NO: 18 - DNA, 24 bp, Artificial
  Synthetic probe sequence
cttcccacgactcacctaacctcg

SEQ ID NO: 19 - DNA, 22 bp, Artificial
  Synthetic primer sequence
tggcgataggattttgttgtga

SEQ ID NO: 20 - DNA, 21 bp, Artificial
  Synthetic primer sequence
gacaacctcccgcaactcttt

SEQ ID NO: 21 - DNA, 22 bp, Artificial
  Synthetic probe sequence
tttcctccgtttcccgaacgaa

SEQ ID NO: 22 - DNA, 22 bp, Artificial
  Synthetic primer sequence
aacgaacgcgcaaaatactaca

SEQ ID NO: 23 - DNA, 28 bp, Artificial
  Synthetic primer sequence
cgttttggtagtttatttcgagattagc

SEQ ID NO: 24 - DNA, 21 bp, Artificial
  Synthetic probe sequence
ccgacgctccgccaacgatta

SEQ ID NO: 25 - DNA, 19 bp, Artificial
  Synthetic primer sequence
cgcacattcgctcaaatcc

SEQ ID NO: 26 - DNA, 22 bp, Artificial
  Synthetic primer sequence
cgtcgagattcgatcgtaggtt

SEQ ID NO: 27 - DNA, 29 bp, Artificial
  Synthetic probe sequence
catccgaacatccacctaaccctaaaacg

SEQ ID NO: 28 - DNA, 21 bp, Artificial
  Synthetic primer sequence
ttcgtcgggtaagcgtttgta

SEQ ID NO: 29 - DNA, 20 bp, Artificial
  Synthetic primer sequence
gcgaccgccatcttacgtaa

SEQ ID NO: 30 - DNA, 26 bp, Artificial
  Synthetic probe sequence
cggcgttgtcgtaaattaggcggttt

SEQ ID NO: 31 - DNA, 28 bp, Artificial
  Synthetic primer sequence
tctaacaattataaactccaaccaccaa

SEQ ID NO: 32 - DNA, 25 bp, Artificial
  Synthetic primer sequence
gggaagatgggatagaagggaatat

SEQ ID NO: 33 - DNA, 36 bp, Artificial
  Synthetic probe sequence
ccttcattctaacccaatacctatcccacctctaaa

SEQ ID NO: 34 - DNA, 23 bp, Artificial
  Synthetic primer sequence
tccgtaagtgcagcttctctttg

SEQ ID NO: 35 - DNA, 20 bp, Artificial
  Synthetic primer sequence
tggagcccacaactgtcaga

SEQ ID NO: 36 - DNA, 34 bp, Artificial
  Synthetic probe sequence
caaagtacagagtcaagagttccaaagccacaga

SEQ ID NO: 37 - RNA, 19 nt, Artificial
  Synthetic primer sequence
gagccuacuuggauguuaa

SEQ ID NO: 38 - RNA, 19 nt, Artificial
  Synthetic primer sequence
uuaacauccaaguaggcuc

SEQ ID NO: 39 - DNA, 20 bp, Artificial
  Synthetic primer sequence
acaggctacgtgctccagat

SEQ ID NO: 40 - DNA, 20 bp, Artificial
  Synthetic primer sequence
ctcacaggccacctgcttat

SEQ ID NO: 41 - DNA, 21 bp, Artificial
  Synthetic primer sequence
cctcatatccgggggaatgtg

SEQ ID NO: 42 - DNA, 21 bp, Artificial
  Synthetic primer sequence
gcagcagctggcaccttattg

SEQ ID NO: 43 - DNA, 22 bp, Artificial
  Synthetic primer sequence
cagaggagcccaagccaaagag

SEQ ID NO: 44 - DNA, 21 bp, Artificial
  Synthetic primer sequence
cacagccgtcccagtcacagt